(19) United States
(12) Patent Application Publication
(10) Pub. No.: US 2013/0267571 A1
(43) Pub. Date: Oct. 10, 2013

(54) TERPENOID ANALOGUES AND USES THEREOF FOR TREATING NEUROLOGICAL CONDITIONS

(71) Applicants: Mark A. Reed, Hubley (CA); Donald Weaver, Halifax (CA); Shengguo Sun, Halifax (CA); Alexander McLellan, Ketch Harbour (CA); Erhu Lu, Halifax (CA)

(72) Inventors: Mark A. Reed, Hubley (CA); Donald Weaver, Halifax (CA); Shengguo Sun, Halifax (CA); Alexander McLellan, Ketch Harbour (CA); Erhu Lu, Halifax (CA)

(73) Assignee: NeuroQuest Inc., Halifax (CA)

(21) Appl. No.: 13/822,826

(22) PCT Filed: Sep. 14, 2011
(86) PCT No.: PCT/CA11/50562

§ 371 Date: Jun. 28, 2013

Related U.S. Application Data
(60) Provisional application No. 61/382,635, filed on Sep. 14, 2010.

(51) Int. Cl. C07C 233/09; C07C 43/166; C07C 57/03; C07D 257/04; C07C 233/65; C07C 233/05; C07C 233/07; C07C 235/60; C07C 43/15; C07C 211/21

(52) U.S. Cl. CPC C07C 233/09; C07C 43/15; C07C 43/166; C07C 57/03; C07C 211/21; C07C 233/65; C07C 233/05; C07C 233/07; C07C 235/60; C07D 257/04
USPC 514/381; 568/687; 514/722; 568/626; 514/717; 562/598; 514/557; 564/204; 514/627; 564/509; 514/671; 564/187; 514/617; 564/215; 514/629; 564/207; 564/177; 514/622; 548/250

(57) ABSTRACT

The present application provides a terpene analogue of Formula (I) or a pharmaceutically acceptable isomer, salt or ester thereof, and methods and uses thereof for treating neurological conditions such as pain in general and neuropathic pain. These terpene analogues can also be used to treat other electrical disorders in the central and peripheral nervous system. Also provided are methods of synthesizing the terpene analogues of Formula I.

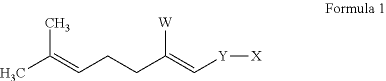

### CROSS-REFERENCE TO RELATED APPLICATIONS/INCORPORATION BY REFERENCE STATEMENT

This application claims benefit as a US national stage application submitted under 35 USC 371 of PCT/CA2011/050562 filed Sep. 14, 2011, which claims the benefit of and priority to U.S. provisional patent application No. 61/382,635, filed Sep. 14, 2010. The above-referenced patent applications are expressly incorporated herein in their entirety as though set forth explicitly herein.

### FIELD OF THE INVENTION

The present application relates to the field of neurological disorders. More specifically, the present application relates to terpenoid analogues and uses thereof for treating pain.

### BACKGROUND

Chronic pain, whether nociceptive or neuropathic, is subject to intensive research, with significant resources being devoted to the development of analgesic drugs. Neuropathic pain is notoriously difficult to treat. Current treatments of neuropathic pain include the use of anti-convulsants, anti-depressants, and opioids. They are often either ineffective or result in unacceptable side effects at the doses required for analgesia. A chronic progressive condition that strikes a generally middle aged and older demographic, neuropathic pain rates are expected continue to rise much higher than the current estimate of more than 12 million present day sufferers in North America alone. The chronic pain associated with peripheral neuropathy is known to result in tremendous human suffering, including loss of mobility, lost productivity, difficulty maintaining social and family relationships, and depression. Therefore there is an unmet medical need for the development of novel treatments for neuropathic pain.

Neuropathic pain is produced by damage to, or pathological changes in, the peripheral central nervous system, typically producing pain that is described as “burning”, “electric”, “tingling”, and “shooting” in nature. Other characteristics of neuropathic pain include hyperpathia, hyperesthesia, dysesthesia, and paresthesia.

Voltage-gated sodium channels in sensory neurons play an essential role in several chronic pain neuropathies that arise from injury to peripheral nerves, such as those caused by trauma, nerve compression, diabetic neuropathy, viral infections or chemotherapeutic agents. Compounds that exhibit a use-dependent blockade of these channels, including anti-convulsants, anti-arrhythmics, local anaesthetics, anti-epilepsy drugs, drugs for sleep disorders, anti-migraine drugs and anti depressants, have been found to be effective in the treatment of neuropathic pain and electrical disorders in the central and peripheral nervous system, which in turn provides clinical support for the importance of these channels in such pain states.

Current conventional pharmacological strategies for treating neuropathic pain include sodium channel blockers, tri-cyclic antidepressants, serotonin reuptake inhibitors, anticonvulsants, GABA B receptor inhibitors, NMDA receptor antagonists, and topical agents. TRP (Transient Receptor Potential Vanilloid) antagonists prevent pain by silencing a nociceptor in the periphery where pain is generated. Compounds that act upon the TRP family of receptors can also be used to treat other electrical disorders in the central and peripheral nervous system.

The efficacy of these pharmacological treatments is often limited by side effects at the doses required for analgesia, as well as in some cases long delays before the onset of analgesia, a substantial rate of nonresponsiveness to therapy, and a potential for addiction. Therefore, there is a need for a novel preparation to treat neuropathic pain.

In terms of inhibition of nerve function, a variety of classes of naturally derived compounds has shown the ability to inhibit neuronal firing by various methods, including affects on nerve cell receptors and associated ion channels. For example, flavanoids, terpenes, terpenoids, ginsenosides, and a variety of other dietary and environmental compounds have been shown to influence nerve transmission rates.

Stotz et al. describe a role of citral and the isolated aldehyde and alcohol cis or trans isomers of citral (neral, nerol, geranial, geraniol) as being effective antagonists of TRP ion channels (Stotz et al., Citral Sensing by Transient Receptor Potential Channels in Dorsal Root Ganglion Neurons. PLoS ONE (2008), 3(5): e2082).

There remains a need for alternative therapies for treating disorders of nerve cell transmission and, in particular, neuropathic pain.

This background information is provided for the purpose of making known information believed by the applicant to be of possible relevance to the presently disclosed and claimed inventive concept(s). No admission is necessarily intended, nor should be construed, that any of the preceding information constitutes prior art against the presently disclosed and claimed inventive concept(s).

### BRIEF DESCRIPTION OF THE FIGURES

FIG. 1 shows a sodium channel patch clamp assay.

FIG. 2 illustrates Ca²⁺ imaging of NQ 2983 at various concentrations in the presence of HEK-TRPV cells.

FIG. 3 shows a dose response curve of a zebrafish embryo assay.

### DETAILED DESCRIPTION

Unless defined otherwise, all technical and scientific terms used herein have the same meaning as commonly understood by one of ordinary skill in the art to which the presently disclosed and claimed inventive concept(s) belongs.

It should also be noted that if the stereochemistry of a structure or a portion of a structure is not indicated with, for example, bold or dashed lines, the structure or the portion of the structure is to be interpreted as encompassing all stereoisomers of it. Curved or “squiggled” bond lines in structures or portions thereof are to be interpreted to encompass all cis and trans stereoisomers. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

More particularly, it should be understood that any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

As used herein, “neuropathic pain” refers to pain caused by various types of nerve damage. Some examples of neuropathic pain conditions that can be treated by the method of the presently disclosed and claimed inventive concept(s) include, but are not limited to, diabetic peripheral neuropathy, herpes zoster, post herpetic neuralgia, trigeminal neuralgia, complex regional pain syndrome, reflex sympathetic dystrophy, migraine headache, phantom limb syndrome, neuropathic pain due to chronic disease (multiple sclerosis, HIV, etc), neuropathic pain due to trauma (causalgia), neuropathic pain due to impingement (i.e., sciatica, carpal tunnel, etc.), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic pain due to fibromylagia, and neuropathic pain with no known cause (idiopathic).

As used herein, a “terpene compound” refers to a terpene, a terpenoid, or a pharmaceutically acceptable isomer, salt, ester or solvate thereof. Isomers can include, for example, (Z)- or (E)-isomers of the terpene compound.

As used herein, a “terpenoid” refers to a chemically modified terpene. Examples of terpenoids include, but are not limited to, terpenoid aldehydes, terpenoid acids, terpenoid esters and terpenoid oxides.

As used herein, a “terpene analogue” is a compound that is an analogue of a terpene compound or a terpenoid, since it is structurally and functionally similar to a terpene compound or terpenoid.

As used herein, “alkyl” means a monovalent straight, branched, or cyclic hydrocarbon radical, e.g., CfH2f+1, where f is an integer, which may include one or more heteroatoms. For example, an alkyl is a C1-C20 monovalent straight, branched, or cyclic hydrocarbon radical. The term “alkyl” encompasses cycloalkyl, heteroalkyl and heterocyclyl moieties. “Alkenyl” means a hydrocarbon moiety that is linear, branched or cyclic and comprises at least one carbon to carbon double bond, which may include one or more heteroatoms. “Alkynyl” means a hydrocarbon moiety that is linear, branched or cyclic and comprises at least one carbon to carbon triple bond, which may include one or more heteroatoms.

“Aryl” means a moiety including a substituted or unsubstituted aromatic ring, including heteroaryl moieties and moieties with more than one conjugated aromatic ring; optionally it may also include one or more non-aromatic ring. “C5 to C8 Aryl” means a moiety including a substituted or unsubstituted aromatic ring having from 5 to 8 carbon atoms in one or more conjugated aromatic rings. Examples of aryl moieties include phenyl.

“Alkylene” means a substituted or unsubstituted divalent alkyl radical, e.g., —CfH2f- wherein f is an integer. “Alkenylene” means a divalent alkenyl radical, e.g., —CHCH—. An alkylene may include one or more heteroatoms. For example, an “alkylene” is a C1-C20 divalent straight, branched, or cyclic hydrocarbon.

“Heterocyclyl” means a moiety including a substituted or unsubstituted cyclic radical having from 2 to 8 carbon atoms and at least one heteroatom in one or more rings. As used herein, “heteroatom” refers to non-carbon and non-hydrogen atoms, such as, for example, O, S, and N. Examples of non-aromatic heterocyclic moieties include imidazolidinyl, pyrazolidinyl, oxazolidinyl and dioxanyl. Included in the term “heterocyclyl” are “heteroaryl” moieties. “Heteroaryl” means a moiety including a substituted or unsubstituted aromatic ring having from 3 to 8 carbon atoms and at least one heteroatom in one or more conjugated aromatic rings. Examples of heteroaryl moieties include pyridyl, furanyl, thienyl, imidazolyl, pyrazolyl, thiazolyl, isothiazolyl, oxadiazolyl.

“Substituted” means having one or more substituent moieties whose presence does not interfere with the desired function or reactivity. Examples of substituents include alkyl, alkenyl, alkynyl, cycloalkyl, aryl, heteroaryl, hydroxyl, alkoxyl, amino, alkylamino, alkenylamino, amide, thioether, alkylcarbonyl, alkylcarbonyloxy, alkoxycarbonyloxy, carbonate, alkoxycarbonyl, aminocarbonyl, alkylthiocarbonyl, halo (such as fluoro, chloro or bromo), acylamino, imino, sulfhydryl, alkylthio, thiocarboxylate, dithiocarboxylate, sulfate, sulfato, sulfonate, sulfamoyl, sulfonamide, nitro, nitrile, azido, heterocyclyl, ether, ester, thioester, or a combination thereof. The substituents may themselves be substituted. For instance, an amino substituent may itself be mono or independently disubstituted by further substituents defined above, such as alkyl, alkenyl, alkynyl, and cycloalkyl.

As used herein, the term “composition” can refer to a pharmaceutical preparation containing a terpene analogue alone. The pharmaceutical composition can be prepared using standard, well-known techniques. Pharmaceutical compositions described herein do not necessarily require inclusion of any pharmaceutically acceptable diluent or excipient. However, such diluents or excipients can be incorporated into the composition as required depending on the desired characteristics of the composition.

An object of the presently disclosed and claimed inventive concept(s) is to provide terpenoid analogues and uses thereof for treating neurological conditions such as pain in general and neuropathic pain specifically. Compounds that show utility for pain can also often be used to treat other electrical disorders in the central and peripheral nervous system.

In accordance with one aspect, there is provided a method of treating a neurological condition comprising administering to a human or animal a therapeutically effective amount of a terpene analogue of Formula I:

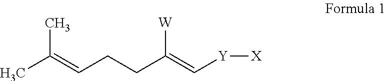

or a pharmaceutically acceptable isomer, salt, or ester thereof, wherein Y is a substituted or unsubstituted C₁ to C₂₀ alkylene, C═O, SO, SO₂, or absent; X is H, OR′, N—(R²)₂, a substituted or unsubstituted C₁ to C₂₀ alkyl, or a substituted or unsubstituted heterocyclyl (for example, heteroaryl), wherein when Y is absent X is not H; R¹ is H, a substituted or unsubstituted C₁ to C₂₀ alkyl, or a substituted or unsubstituted CH₂-aryl; each R² is independently H, a substituted or unsubstituted C₁ to C₂₀ alkyl, aryl, OR¹, CN or C(═O)—R³; R³ is a substituted or unsubstituted C₁ to C₂₀ alkyl, or a substituted or unsubstituted aryl; and W is H, a substituted or unsubstituted C₁ to C₂₀ alkyl, or a substituted or unsubstituted aryl.

In accordance with another aspect, there is provided a use of a terpene analogue for treating a neurological condition in a human or animal, wherein the terpene analogue is defined by Formula 1:

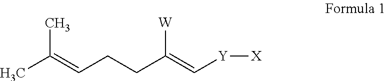

or a pharmaceutically acceptable isomer, salt, or ester thereof, wherein Y is a substituted or unsubstituted C₁ to C₂₀ alkylene, C═O, SO, SO₂, or absent; X is H, OR¹, N—(R²)₂, a substituted or unsubstituted C₁ to C₂₀ alkyl, or a substituted or unsubstituted heterocyclyl (for example, heteroaryl), wherein when Y is absent X is not H; R¹ is H, a substituted or unsubstituted C₁ to C₂₀ alkyl, or a substituted or unsubstituted CH₂-aryl; each R² is independently H, a substituted or unsubstituted C₁ to C₂₀ alkyl, aryl, OR¹, CN or C(═O)—R³; R³ is a substituted or unsubstituted C₁ to C₂₀ alkyl, or a substituted or unsubstituted aryl; and W is H, a substituted or unsubstituted C₁ to C₂₀ alkyl, or a substituted or unsubstituted aryl.

In certain embodiments, the terpene analogue is represented by Formula 1a:

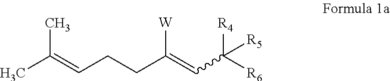

or a pharmaceutically acceptable isomer, salt, or ester thereof, wherein R⁴ is OH, alkoxyl, aryloxyl, —NH₂, —SO₂Aryl, SO₂alkyl, SOalkyl, —SO₂NHAryl, —NHSO₂Aryl, —NHalkyl, —N(alkyl)₂, or —NHCO-Aryl; and W, R⁵, and R⁶ are each independently H, a substituted or unsubstituted C₁ to C₂₀ alkyl, a substituted or unsubstituted aryl or a substituted or unsubstituted alkylaryl. In certain embodiments, the terpene analogue is an isomer, which can be, for example, (Z)- or (E)-isomers of the terpene analogue.

TRP (Transient Receptor Potential Vanilloid) antagonists prevent pain by silencing a nociceptor in the periphery where pain is generated. Surprisingly, the present inventors have found that the terpenoid analogues described herein can be useful for treating disorders of nerve transmission, such as neuropathic pain, by restoring the balance between nerve excitation and inhibition. This can be achieved by affecting the activity of neuronal channels, such as sodium ion channels and TRP.

The present application further provides pharmaceutical compositions for treating neurological conditions, said compositions comprising a terpene analogue of Formula 1:

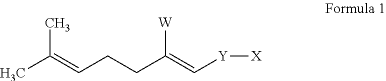

or a pharmaceutically acceptable isomer, salt, or ester thereof, wherein Y is a substituted or unsubstituted C₁ to C₂₀ alkylene, C═O, SO, SO₂, or absent; X is H, OR′, N—(R²)₂, a substituted or unsubstituted C₁ to C₂₀ alkyl, or a substituted or unsubstituted heterocyclyl (for example, heteroaryl), wherein when Y is absent X is not H; R¹ is H, a substituted or unsubstituted C₁ to C₂₀ alkyl, or a substituted or unsubstituted CH₂-aryl; each R² is independently H, a substituted or unsubstituted C₁ to C₂₀ alkyl, aryl, OR¹, CN or C(═O)—R³; R³ is a substituted or unsubstituted C₁ to C₂₀ alkyl, or a substituted or unsubstituted aryl; and W is H, a substituted or unsubstituted C₁ to C₂₀ alkyl, or a substituted or unsubstituted aryl.

In one embodiment, the pharmaceutical composition for treating a neurological condition comprises a terpene analogue of Formula 1a:

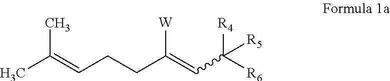

or a pharmaceutically acceptable isomer, salt, or ester thereof, wherein R⁴ is OH, alkoxyl, aryloxyl, —NH₂, —SO₂Aryl, —SO₂alkyl, —SOalkyl, —SO₂NHAryl, —NHSO₂Aryl, —NHalkyl, —N(alkyl)₂, or —NHCO-Aryl; and W, R⁵, and R⁶ are each independently H, a substituted or unsubstituted C₁ to C₂₀ alkyl, a substituted or unsubstituted aryl or a substituted or unsubstituted alkylaryl.

In certain embodiments, the terpene analogue is an isomer, which can be, for example, (Z)- or (E)-isomers of the terpene analogue.

In accordance with another aspect, there is provided a pharmaceutical composition comprising a terpene analogue of Formula 1 or 1a in amount effective to influence the balance between nerve excitation and inhibition following administration to a subject. It has been found that affecting the activity of both sodium gated ion channels and/or TRP channels can be useful in the treatment of disorders of nerve transmission, such as neuropathic pain, by restoring the balance between nerve excitation and inhibition.

The therapeutic terpene analogues described herein can be administered to a subject by a route which is effective for restoring the balance between nerve excitation and inhibition by affecting the activity of both sodium ion channels and TRP channels. Suitable routes of administration include intravenous, topical, oral, intranasal, intravaginal and intrarectal. The terpene analogues can be administered with a pharmaceutically acceptable vehicle.

The compositions of the present application are prepared using isolated or purified terpene analogues, for example, one or more compounds of Formula 1, or corresponding pharmaceutically acceptable salts, esters or solvates thereof as active components. The term “solvate” is intended to include “hydrate”. The compositions of the presently disclosed and claimed inventive concept(s) are not natural oils derived as distillates of plant material; however, the terpene analogues used to prepare such synthetic compositions can include one or more compounds that have been isolated from plant material.

Exemplary terpene analogues include monterpenoid analoguess of 3,7-dimethylocta-2,6-dien-1-ol. These are shown in Table 1.

TABLE 1

| ID | Terpene analogue | | |
|---|---|---|---|
| Number | structure | Properties | Name |
| 2976 | 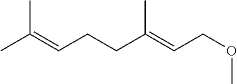 | Chemical Formula: C₁₁H₂₀O Molecular Weight: 168.28 | (E)-1-methoxy-3,7- dimethylocta-2,6-diene |
| 2977 | 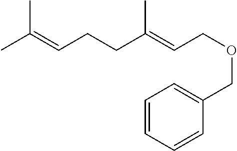 [SMILES: CC(C)=CCC/C(C)=C/COCc1ccccc1] | Chemical Formula: C₁₇H₂₄O Molecular Weight: 244.37 | (E)-((3,7-dimethylocta- 2,6- dienyloxy)methyl)benzene |
| 2978 | 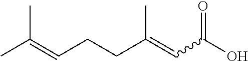 | Chemical Formula: C₁₀H₁₆O₂ Molecular Weight: 168.23 | 3,7-dimethyloct-2,6- dienoic acid |
| 2980 | 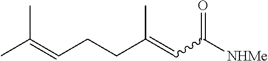 | Chemical Formula: C₁₁H₁₉NO Molecular Weight: 181.27 | N,3,7-trimethylocta-2,6- dienamide |
| 2981 | 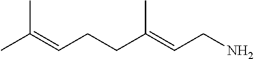 | Chemical Formula: C₁₀H₁₉N Molecular Weight: 153.26 | (E)-3,7-dimethylocta-2,6- dien-1-amine |
| 2982 | 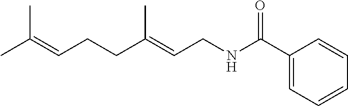 [SMILES: CC(C)=CCC/C(C)=C/CNC(=O)c1ccccc1] | Chemical Formula: C₁₇H₂₃NO Exact Mass: 257.1780 | (E)-N-(3,7-dimethylocta- 2,6-dienyl)benzamide |
| 2983 | 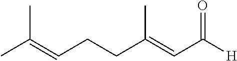 | Chemical Formula: C₁₀H₁₆O Exact Mass: 152.1201 | (E)-3,7-dimethylocta-2,6- dienal |
| 2984 | 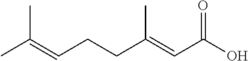 | Chemical Formula: C₁₀H₁₆O₂ Molecular Weight: 168.23 | (E)-3,7-dimethylocta-2,6- dienoic acid |
| 2985 | 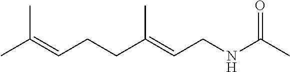 | Chemical Formula: C₁₂H₂₁NO Exact Mass: 195.1623 | (E)-N-(3,7-dimethylocta- 2,6-dienyl)acetamide |
| 2986 | 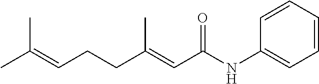 [SMILES: CC(C)=CCC/C(C)=C/C(=O)Nc1ccccc1] | Chemical Formula: C₁₆H₂₁NO Exact Mass: 243.1623 | (E)-3,7-dimethyl-N- phenylocta-2,6-dienamide |
| 2987 | 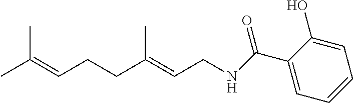 [SMILES: CC(C)=CCC/C(C)=C/CNC(=O)c1ccccc1O] | Chemical Formula: C₁₇H₂₃NO₂ Exact Mass: 273.1729 | (E)-N-(3,7-dimethylocta- 2,6-dienyl)-2- hydroxybenzamide |
| 2988 | 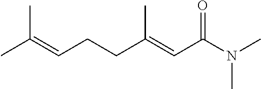 | Chemical Formula: C₁₂H₂₁NO Molecular Weight: 195.301 | (E)-N,N,3,7- tetramethylocta-2,6- dienamide |
| 2990 | 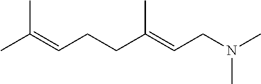 | Chemical Formula: C₁₂H₂₃N Molecular Weight: 181.318 | (E)-N,N,3,7- tetramethylocta-2,6-dien- 1-amine |
| 2991 | 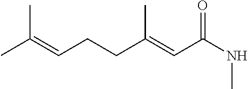 | Chemical Formula: C₁₁H₁₉NO Molecular Weight: 181.275 | (E)-N,3,7-trimethylocta- 2,6-dienamide |
| 2992 | 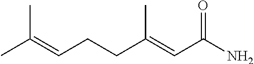 | Chemical Formula: C₁₀H₁₇NO Molecular Weight: 167.248 | (E)-3,7-dimethylocta-2,6- dienamide |
| 3000 | 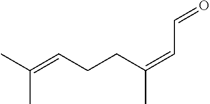 | Chemical Formula: C₁₀H₁₆O Molecular Weight: 152.233 | (Z)-3,7-dimethylocta-2,6- dienal |
| 3001 | 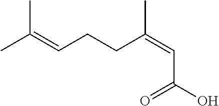 | Chemical Formula: C₁₀H₁₆O₂ Molecular Weight: 168.233 | (Z)-3,7-dimethylocta-2,6- dienoic acid |
| 3007 | 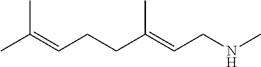 | Chemical Formula: C₁₁H₂₁N Molecular Weight: 167.291 | (E)-N,3,7-trimethylocta- 2,6-dien-1-amine |
| 3045 | 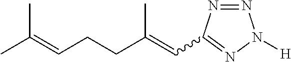 | Chemical Formula: C₁₀H₁₆N₄ Molecular Weight: 192.261 | 5-(2,6-dimethylhepta-1,5- dien-1-yl)-2H-tetrazole |
| 3047 | 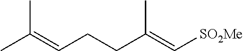 | Chemical Formula: C₁₀H₁₈O₂S Molecular Weight: 202.314 | (E)-2,6-dimethyl-1- (methylsulfonyl)hepta- 1,5-diene |
| 3050 | 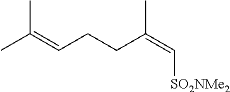 | Chemical Formula: C₁₁H₂₁NO₂S Molecular Weight: 231.355 | (Z)-N,N,2,6- tetramethylhepta-1,5- diene-1-sulfonamide |
| 3051 | 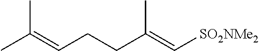 | Chemical Formula: C₁₁H₂₁NO₂S Molecular Weight: 231.355 | (E)-N,N,2,6- tetramethylhepta-1,5- diene-1-sulfonamide |
| 3052 | 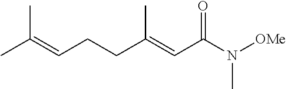 | Chemical Formula: C₁₂H₂₁NO₂ Molecular Weight: 211.301 | (E)-N-methoxy-N,3,7- trimethylocta-2,6- dienamide |
| 3053 | 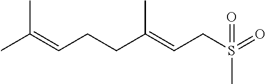 | Chemical Formula: C₁₁H₂₀O₂S Molecular Weight: 216.340 | (E)-3,7-dimethyl-1- (methylsulfonyl)octa-2,6- diene |
| 3054 | 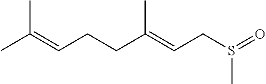 | Chemical Formula: C₁₁H₂₀OS Molecular Weight: 200.341 | (E)-3,7-dimethyl-1- (methylsulfinyl)octa-2,6- diene |
| 3055 | 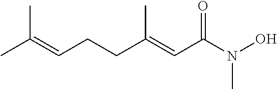 | Chemical Formula: C₁₁H₁₉NO₂ Molecular Weight: 197.274 | (E)-N-hydroxy-N,3,7- trimethylocta-2,6- dienamide |
| 3057 | 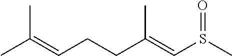 | Chemical Formula: C10H18OS Molecular Weight: 186.314 | (E)-2,6-dimethyl-1- (methylsulfinyl)hepta-1,5- diene |
| 3060 | 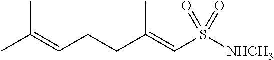 | Chemical Formula: C10H19NO2S Molecular Weight: 217.328 | (E)-N,2,6-trimethylhepta- 1,5-diene-1-sulfonamide |
| 3061 | 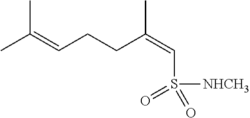 | Chemical Formula: C10H19NO2S Molecular Weight: 217.328 | (Z)-N,2,6-trimethylhepta- 1,5-diene-1-sulfonamide |
| 3062 | 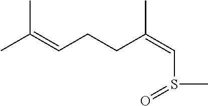 | Chemical Formula: C10H18OS Molecular Weight: 186.314 | (Z)-2,6-dimethyl-1- (methylsulfinyl)hepta-1,5- diene |
| 3063 | 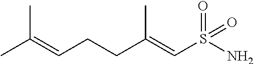 | Chemical Formula: C9H17NO2S Molecular Weight: 203.302 | (E)-2,6-dimethylhepta- 1,5-diene-1-sulfonamide |
| 3064 | 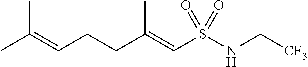 [SMILES: CC(C)=CCC/C(C)=C/S(=O)(=O)NCC(F)(F)F] | Chemical Formula: C11H18F3NO2S Molecular Weight: 285.326 | (E)-2,6-dimethyl-N- (2,2,2- trifluoroethyl)hepta-1,5- diene-1-sulfonamide |
| 3065 | 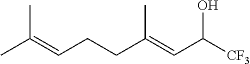 | Chemical Formula: C11H17F3O Molecular Weight: 222.247 | (E)-1,1,1-trifluoro-4,8- dimethylnona-3,7-dien-2- ol |
| 3066 | 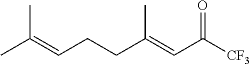 | Chemical Formula: C11H15F3O Molecular Weight: 220.231 | (E)-1,1,1-trifluoro-4,8- dimethylnona-3,7-dien-2- one |
| 3067 | 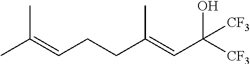 [SMILES: CC(C)=CCC/C(C)=C/C(O)(C(F)(F)F)C(F)(F)F] | Chemical Formula: C12H16F6O Molecular Weight: 290.245 | (E)-1,1,1-trifluoro-4,8- dimethyl-2- (trifluoromethyl)nona-3,7- dien-2-ol |
| 3069 | 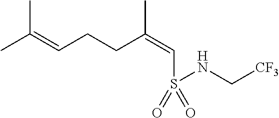 [SMILES: CC(C)=CCC/C(C)=C\S(=O)(=O)NCC(F)(F)F] | Chemical Formula: C11H18F3NO2S Molecular Weight: 285.326 | (Z)-2,6-dimethyl-N- (2,2,2- trifluoroethyl)hepta-1,5- diene-1-sulfonamide |
| 3070 | 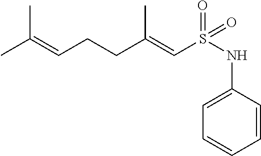 [SMILES: CC(C)=CCC/C(C)=C/S(=O)(=O)Nc1ccccc1] | Chemical Formula: C15H21NO2S Molecular Weight: 279.398 | (E)-2,6-dimethyl-N- phenylhepta-1,5-diene-1- sulfonamide |
| 3071 | 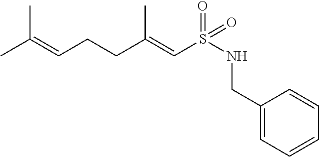 [SMILES: CC(C)=CCC/C(C)=C/S(=O)(=O)NCc1ccccc1] | Chemical Formula: C16H23NO2S Molecular Weight: 293.424 | (E)-N-benzyl-2,6- dimethylhepta-1,5-diene- 1-sulfonamide |
| 3078 | 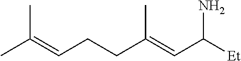 | Chemical Formula: C12H23N Molecular Weight: 181.318 | (E)-5,9-dimethyldeca-4,8- dien-3-amine |
| 3079 | 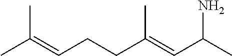 | Chemical Formula: C11H21N Molecular Weight: 167.291 | (E)-4,8-dimethylnona-3,7- dien-2-amine |
| 3081 | 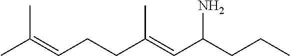 | Chemical Formula: C13H25N Molecular Weight: 195.344 | (E)-6,10-dimethylundeca- 5,9-dien-4-amine |
| 3082 | 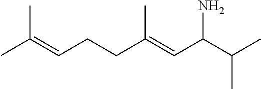 | Chemical Formula: C13H25N Molecular Weight: 195.344 | (E)-2,5,9-trimethyldeca- 4,8-dien-3-amine |
| 3083 | 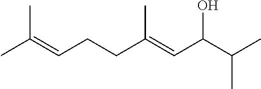 | Chemical Formula: C13H24NO Molecular Weight: 196.329 | (E)-2,5,9-trimethyldeca- 4,8-dien-3-ol |
| 3084 | 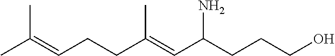 | Chemical Formula: C13H25NO Molecular Weight: 211.344 | (E)-4-amino-6,10- dimethylundeca-5,9-dien- 1-ol |
| 3085 | 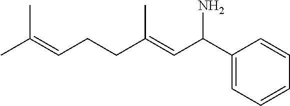 [SMILES: CC(C)=CCC/C(C)=C/C(N)c1ccccc1] | Chemical Formula: C16H23N Molecular Weight: 229.361 | (E)-3,7-dimethyl-1- phenylocta-2,6-dien-1- amine |
| 3089 | 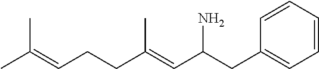 [SMILES: CC(C)=CCC/C(C)=C/C(N)Cc1ccccc1] | Chemical Formula: C17H25N Molecular Weight: 243.387 | (E)-4,8-dimethyl-1- phenylnona-3,7-dien-2- amine |

The compositions of the present application can be prepared and administered in a wide variety of dosage forms, such as, but not limited to, compositions in the form of a suspension, pill, gel, oil, cream, patch, spray or aerosol. The composition can be formulated to be suitable for oral administration, topical administration, intranasal, transdermal, intravaginal, and intrarectal administration. Processes for manufacture of such compositions are briefly described below; however, the techniques employed in these processes are standard and well known to a worker skilled in the art. It will be obvious to those skilled in the art that the following dosage forms can comprise as the active component, a compound of Formula 1 or 1a, a corresponding pharmaceutically acceptable salt, ester or solvate thereof, or any combination thereof.

For preparing pharmaceutical compositions from the terpene analogues of Formula 1 or 1a, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. Liquid preparations for parenteral injection can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

A particular mode of administration of the composition of the present application is to a skin surface via a topical route. Such a composition is topically applied in the form of a lotion, solution, cream, ointment or powder. For example, the composition can be formulated into a cream consisting of an aqueous emulsion of polyethylene glycols or liquid paraffin or can be incorporated at a concentration between 1 and 10% into an ointment consisting of a white wax or white soft paraffin base together with such stabilizers and preservatives as may be required. The topical compositions can contain additional ingredients such as binders, excipients, antioxidants, and dyes.

The pharmaceutical preparation may be provided in unit dosage form. In such form the preparation is subdivided into unit doses containing appropriate quantities of the active component. The unit dosage form can be a packaged preparation, the package containing discrete quantities of preparation, such as packeted creams, lotions, ointments, tablets, capsules, or powders in tubes, vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

To gain a better understanding of the presently disclosed and claimed inventive concept(s) described herein, the following examples are set forth. It should be understood that these examples are for illustrative purposes only. Therefore, they should not limit the scope of this presently disclosed and claimed inventive concept(s) in any way.

### EXAMPLES

The activity of the terpene analogues of the presently disclosed and claimed inventive concept(s), including their ability to affect nerve transmission, can be evaluated using different assays known in the art. For example, assays which may be particularly useful include the sodium channel patch clamp, the zebrafish anaesthesia assay, and/or a TRPV1 assay.

a) Sodium Channel—Changes in neuronal excitability as a result of alteration of ion channel activity and/or function by a bioactive substance can be examined using typical slices taken from the rodent brain or spinal cord.

b) Zebrafish Anaesthesia Assay—The zebrafish (Danio rerio) model organism is increasingly used for assessing drug toxicity and safety. Numerous studies now confirm that mammalian and zebrafish toxicity profiles are strikingly similar. We have found, using a tailored Zebrafish assay, that this assay is a vertebrate model which can be utilized as a screening tool for analgesic activity.

c) TRPV1 Assay—TRPV1 (Transient Receptor Potential Vanilloid, Type 1) is a member of the transient receptor potential (TRP) family of ion channels. These channels mediate numerous sensory interactions, including nociception, inflammation, and their modulation is useful in a number of related pathologies, pain being one example. Thus, modulation of TRPV1 is therefore an attractive prospect for drug development in the field of analgesia. Because TRP channels are selective for calcium ions, the uptake of Ca²⁺ provides a basis for the development of a functional assay to assess ligand potency.

### Example 1

The following examples were synthesised according to Scheme 1:

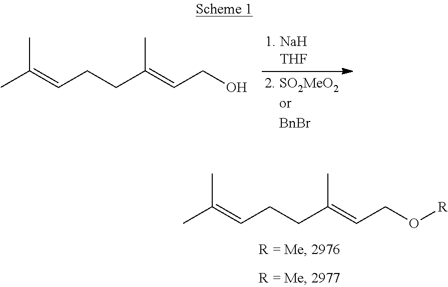

### NQ 2976(E)-1-methoxy-3,7-dimethylocta-2,6-diene

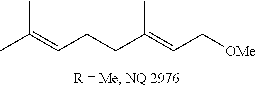

To a suspension of sodium hydride (1.56 g, 0.038mol, 60% dispersion in mineral oil) in NMP 25 mL was added at 0° C. a solution of geraniol (5 g, 0.032 mol) in NMP (25 mL). Upon complete addition the cooling bath was removed and the solution was stirred for 1 h then recooled to 0° C. To the reaction was then added dimethyl sulphate (4.65 mL, 0.048 mol) dropwise. The reaction was stirred for 16 h then quenched with water (100 mL), extracted with hexanes (3×30 mL), washed with brine (10 mL), dried (Na₂SO₄), filtered and then concentrated in vacuo to give (E)-1-methoxy-3,7-dimethylocta-2,6-diene as colorless oil (5.2 g, 0.031 mol).

The spectral data are as follows: ¹H NMR (500 MHz, CDCl₃) δ (ppm) 1.65 (m, 6H), 1.63 (s, 3H), 2.15 (m, 4H), 3.4 (s, 3H), 3.95 (m, 2H), 5.1 (m, 1H), 5.4 (m, 1H).

NQ 2977:

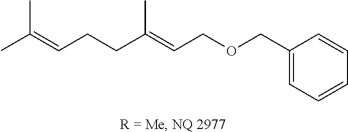

To a suspension of sodium hydride (1.56 g, 0.038 mol, 60% dispersion in mineral oil) in NMP 25 mL was added at 0° C. a solution of geraniol (5 g, 0.032 mol) in NMP (25 mL). Upon complete addition the cooling bath was removed and the solution was stirred for 1 h then recooled to 0° C. To the reaction was then added benzyl bromide (5.2 mL, 0.038 mol) dropwise. The reaction was stirred for 16 h then quenched with water (100 mL), extracted with hexanes (3×30 mL), washed with brine (10 mL), dried (Na₂SO₄), filtered and then concentrated in vacuo to give (E)-((3,7-dimethylocta-2,6-dienyloxy)methyl)benzene as colorless oil (8.29 g, 0.030 mol)

The spectral data are as follows: ¹H NMR (500 MHz, CDCl₃) δ (ppm) 1.69 (m, 9H), 2.15 (m, 4H), 4.05 (m, 2H), 4.73 (s, 2H), 7.1-7.3 (m, 5H).

NQ2978:

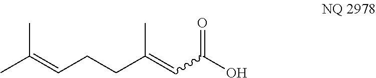

Purchased from Fluka (a division of Aldrich and Co as a mixture of cis/trans isomers (Fluka catalogue number: 48813, Geranic acid, technical grade, mixture of isomers, ˜85% GC)

### Example 2

The following examples were synthesized according to Scheme 2:

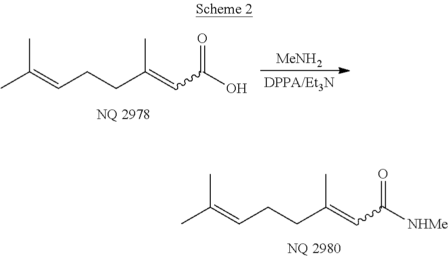

NQ 2980:

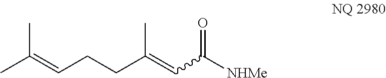

To a solution of (E)-3,7-dimethylocta-2,6-dienoic acid (0.50 g, 3.0 mmol), methylamine solution (3.0 mL, 6.0 mmol, 2 M) and triethylamine (2.50 mL, 17.8 mmol) in THF (15 mL) was added DPPA (0.70 mL, 3.3 mmol) and stirred for 16 hours. The mixture was quenched with water (10 mL) and extracted with ethyl acetate (2×20 ml). The organic phase was dried (sodium sulphate), concentrated in vacuum then subjected to flash column chromatography (50% acetyl acetate in hexanes) to furnish (E)-N,3,7-trimethylocta-2,6-dienamide (0.40 g, 74%).

The spectral data for NQ 2980 are as follows: ¹H NMR (500 MHz, CDCl₃) δ (ppm) 1.63 (s, 3H), 1.71 (s, 3H), 2.10 (m, 7H), 2.87 (s, 3H), 5.11 (t, J=6.7 Hz, 1H), 5.56 (s, 1H), 5.57 (m, 1H); ¹C NMR (125 MHz, CDCl₃): δ (ppm) 18.2, 18.7, 26.1, 26.6, 41.2, 118.3, 123.7, 132.8, 154.3, 168.3.

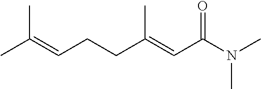

NQ 1013:

To a solution of 0.50 g (3.0 mmol) geranic acid and 4.2 ml (30.0 mmol) triethylamine in 20 ml dry THF added 1.2 g (14.9 mmol) dimethylamine hydrochloride at room temperature. 0.64 ml (3.0 mmol) DPPA was added after 10 min. The reaction was stirred overnight and quenched with 10 ml water, followed by extraction with ethyl acetate (2×20 ml). The extraction was dried over anhydrous sodium sulfate before evaporation. (E)-N,N,3,7-tetramethylocta-2,6-dienamide (0.40 g, 70%) was obtained by flash column chromatography (50% acetyl acetate in hexanes).

The spectral data are as follows: ¹H NMR (500 MHz, CDCl₃) δ (ppm) 1.64 (s, 3H), 1.71 (s, 3H), 1.91 (s, 3H), 2.14 (m, 4H), 3.00 (s, 3H), 3.03 (s, 3H), 5.12 (m, 1H), 5.80 (d, J=0.9 Hz, 1H); ¹³C NMR (125 MHz, CDCl₃): δ (ppm) 18.2, 18.9, 26.2, 26.4, 35.1, 38.1, 40.1, 118.4, 124.0, 132.6, 148.9, 169.3.

NQ 1016

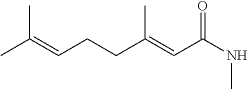

To a solution of 0.50 g (3.0 mmol) geranic acid and 4.2 ml (30.0 mmol) triethylamine in 20 ml dry THF added 1.0 g (15.6 mmol) methylamine hydrochloride at room temperature. 0.64 ml (3.0 mmol) DPPA was added after 10 min. The reaction was stirred overnight and quenched with 10 ml water, followed by extraction with ethyl acetate (2×20 ml). The extraction was dried over anhydrous sodium sulfate before evaporation. (E)-N,3,7-trimethylocta-2,6-dienamide (0.40 g, 75%) was obtained by flash column chromatography (50% acetyl acetate in hexanes).

The spectral data are as follows: ¹H NMR (500 MHz, CDCl₃) δ (ppm) 1.67 (s, 3H), 1.72 (s, 3H), 2.15 (m, 7H), 2.87 (s, 3H), 5.10 (m, 1H), 5.56 (s, br, 1H), 5.57 (s, 1H); ¹³C NMR (125 MHz, CDCl₃): δ (ppm) 18.2, 18.7, 26.1, 26.4, 26.6, 41.2, 118.3, 123.7, 132.8, 154.3, 168.3.

NQ 1017:

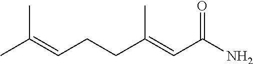

To a solution of 0.50 g (3.3 mmol) NQ 1009 and 2.1 ml (14.9 mmol) triethylamine in 20 ml dry THF added 1.13 g HATU. After 10 min, 2.0 ml (14.9 mmol) 7 N ammonia in methanol was added at room temperature. The reaction was stirred overnight and quenched with 10 ml water, followed by extraction with ethyl acetate (2×20 ml) and washed with water (2×10 ml). The extraction was dried over anhydrous sodium sulfate before evaporation. (E)-3,7-dimethylocta-2,6-dienamide (0.32 g, 60%) was obtained by flash column chromatography (60% acetyl acetate in hexanes).

The spectral data are as follows: ¹H NMR (500 MHz, CDCl₃) δ (ppm) 1.65 (s, 3H), 1.72 (s, 3H), 2.15 (m, 7H), 5.11 (m, 1H), 5.45 (s, br, 2H), 5.64 (s, 1H); ¹³C NMR (125 MHz, CDCl₃): δ (ppm) 18.2, 18.7, 26.1, 26.5, 41.3, 117.3, 123.5, 132.9, 156.6, 169.5.

### Example 4

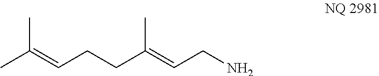

This compound was purchased from Aldrich as a single isomer; catalogue number: 412643 Aldrich Geranylamine, single isomer, 90%.

### Example 5

The following examples were synthesized according to Scheme 3:

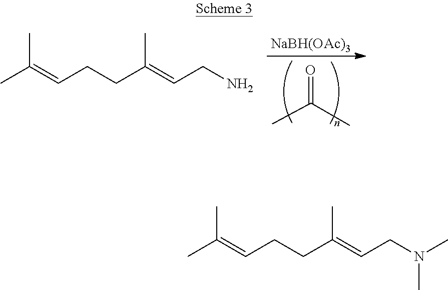

NQ 1015:

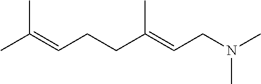

To a suspension of 0.5 g (3.2 mmol) geranylmine and 0.48 g (16.0 mmol) paraformaldehyde in 30 ml dry dichloromethane was added 1 ml acetic acid under argon atmosphere. The suspension was stirred for 2 hours before adding 2.7 g (12.8 mmol) sodium triacetoxylborohydride. The reaction was stirred overnight before quenching with 20 ml water. The mixture was extracted with ethyl acetate (2×20 ml) and dried over anhydrous sodium sulfate. (E)-N,N,3,7-tetramethylocta-2,6-dien-1-amine (0.12 g, 21%) was obtained by flash column chromatography (1% triethylamine in acetyl acetate).

The spectral data are as follows: ¹H NMR (500 MHz, CDCl₃) δ (ppm) 1.63 (s, 3H), 1.71 (s, 3H), 1.75 (s, 3H), 2.62 (s, 6H), 2.18 (m, 4H), 3.52 (d, J=8.0 Hz, 2H), 5.07 (m, 1H), 5.37 (dt, J=8.0, 1.1 Hz, 1H); ¹³C NMR (125 MHz, CDCl₃): δ (ppm) 17.1, 18.2, 26.2, 26.5, 40.4, 48.9, 49.0, 60.2, 113.9, 123.9, 132.8, 147.6.

### Example 6

The following examples were synthesised according to Scheme 4:

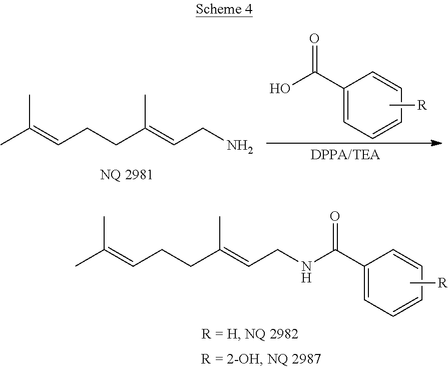

NQ 2982:

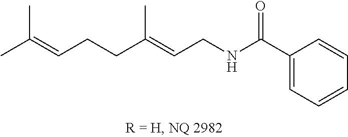 [SMILES: CC(C)=CCC/C(C)=C/CNC(=O)c1ccccc1]

To a solution of benzoic acid (0.32 g, 2.6 mmol), (E)-3,7-dimethylocta-2,6-dien-1-amine (0.24 ml, 1.3 mmol) and triethylamine (1.1 mL, 7.8 mmol) in THF (15 mL) was added DPPA (0.37 mL, 1.7 mmol) and the reaction was stirred for 16 hours. The mixture was quenched with water (10 mL) and extracted with ethyl acetate (2×20 ml). The organic phase was dried (sodium sulphate), concentrated in vacuum then subjected to flash column chromatography (20% acetyl acetate in hexanes), to furnish (E)-N-(3,7-dimethylocta-2,6-dienyl)benzamide (0.30 g, 89%).

The spectral data for NQ 2982 are as follows: ¹H NMR (500 MHz, CDCl₃) δ (ppm) 1.64 (s, 3H), 1.66 (s, 3H), 1.76 (s, 3H), 2.10 (t, J=7.0 Hz, 2H), 2.13 (m, 2H), 4.10 (dd, J=5.9, 6.3 Hz, 2H), 5.12 (t, J=5.7 Hz, 1H), 5.34 (dt, J=1.1, 7.0 Hz, 1H), 6.03 (s, br, 1H), 7.45-7.54 (m, 3H), 7.79 (d, J=7.1 Hz, 2H); ¹C NMR (125 MHz, CDCl₃): δ (ppm) 16.8, 18.2, 26.2, 26.9, 38.5, 40.0, 120.2, 124.3, 127.3, 129.0, 131.8, 132.3, 135.2, 140.9, 167.8.

NQ 2987:

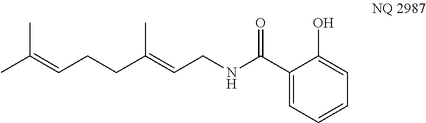 [SMILES: CC(C)=CCC/C(C)=C/CNC(=O)c1ccccc1O]

To a solution of (E)-3,7-dimethylocta-2,6-dien-1-amine (0.50 g, 3.3 mmol) and triethylamine (1.3 ml, 9.8 mmol) in THF (20 mL) was added 2-hydroxybenzoic acid (0.45 mL, 3.3 mmol) followed by DPPA (0.46 ml). The reaction was stirred overnight and quenched with 10 ml water, followed by extraction with ethyl acetate (2×15 ml) and washed with water (2×15 ml). The extraction was dried over anhydrous sodium sulfate before evaporation. (E)-N-(3,7-dimethylocta-2,6-dienyl)-2-hydroxybenzamide (0.30 g, 33%) was obtained by flash column chromatography (20% acetyl acetate in hexanes).

The spectral data for NQ 2987 are as follows: ¹H NMR (500 MHz, CDCl₃) δ (ppm) 1.65 (s, 3H), 1.72 (s, 3H), 1.77 (s, 3H), 2.07-2.17 (m, 4H), 4.08 (dd, J=5.9, 6.3 Hz, 2H), 5.12 (m, 1H), 5.34 (m, 1H), 6.20 (s, 1H), 6.87 (dt, J=1.0, 7.0 Hz, 1H), 7.01 (dd, J=1.0, 8.3 Hz, 1H), 7.37 (dd, J=1.5, 8.1 Hz, 1H), 7.40 (dt, J=1.0, 8.3 Hz, 1H), 12.42 (s, 1H); ¹³C NMR (125 MHz, CDCl₃): δ (ppm) 16.9, 18.2, 26.2, 26.8, 38.1, 40.0, 114.8, 119.0, 119.1, 119.5, 124.2, 125.7, 134.6, 141.7, 162.0, 170.2.

### Example 7

The following examples were synthesised according to Scheme 5:

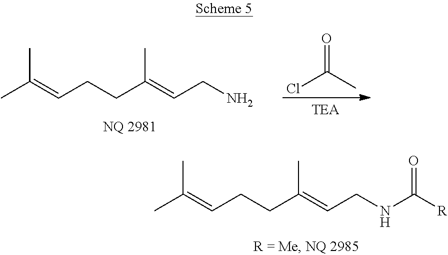

NQ 2985:

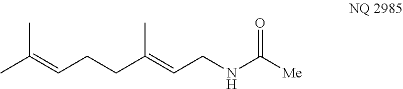

To a solution of (E)-3,7-dimethylocta-2,6-dien-1-amine (0.2 g, 1.3 mmol) and triethylamine (0.55 mL, 4.0 mmol) in THF (15 mL) at 0° C. was added acetyl chloride (0.14 mL, 2.0 mmol). The reaction was stirred for 2 hours and quenched with water (10 mL), extracted with ethyl acetate (2×10 ml) and washed with water (2×10 ml). The organic was dried (sodium sulphate), concentrated in vacuum then subjected to flash column chromatography (65% acetyl acetate in hexanes to furnish (E)-N-(3,7-dimethylocta-2,6-dienyl)acetamide (0.20 g, 80%).

The spectral data for NQ 2985 are as follows: ¹H NMR (500 MHz, CDCl₃) δ (ppm) 1.63 (s, 3H), 1.69 (d, 6H), 2.00 (s, 3H), 2.04 (t, J=7.7 Hz, 2H), 2.13 (m, 2H), 3.88 (t, J=6.1 Hz, 2H), 5.10 (t, J=6.8 Hz, 1H), 5.22 (t, J=7.1 Hz, 1H), 5.44 (s, br, 1H); ¹³C NMR (125 MHz, CDCl₃): δ (ppm) 16.7, 18.1, 23.7, 26.1, 26.9, 38.1, 39.9, 120.3, 124.3, 132.2, 140.5, 170.3.

### Example 8

The following examples were synthesised according to Scheme 6:

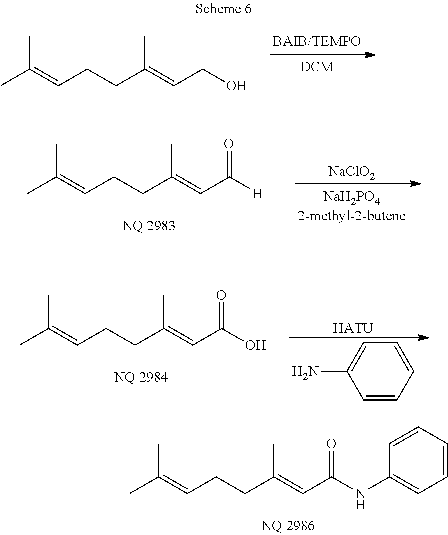

NQ 2983:

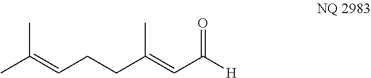

To a solution of (E)-3,7-dimethylocta-2,6-dien-1-ol (3.0 g, 19.5 mmol) in dichloromethane (30 mL) was added [bis(acetoxy)iodo]benzene (6.3 g, 19.5 mmol) and TEMPO (0.3 g, 1.9 mmol). The reaction was stirred for 2 hours then quenched with saturated sodium thiosulfate (10 mL) and extracted with ethyl acetate (3×30 ml). The organic phase was dried, (sodium sulphate) filtered and then concentrated under vacuum. The crude product was subjected to flash column chromatography (10% acetyl acetate in hexanes) to furnish (E)-3,7-dimethylocta-2,6-dienal (2.8 g, 93%).

The spectral data for NQ 2983 are as follows: ¹H NMR (500 MHz, CDCl₃) δ (ppm) 1.72 (s, 3H), 1.73 (s, 3H), 2.21-2.30 (m, 7H), 5.10 (t, J=6.8 Hz, 1H), 5.92 (d, J=8.0 Hz, 1H), 10.0 (d, J=8.0 Hz, 1H); ¹³C NMR (125 MHz, CDCl₃): δ (ppm) 14.0, 17.4, 17.5, 20.9, 25.4, 25.5, 40.2, 122.4, 127.2, 132.8, 163.7, 191.2.

NQ 2984:

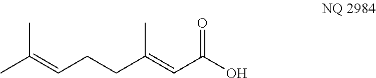

To a solution of (E)-3,7-dimethylocta-2,6-dienal (0.50 g, 3.3 mmol) and 2-methyl-2-butene (3.5 mL, 32.8 mmol) in DMSO (20 mL) was added dropwise sodium chlorite (3.0, 32.8 mmol) and monosodium phosphate (2.8 g, 23.0 mmol) in water (30 ml) at room temperature and stirred for 16 hours. The reaction was extracted with ethyl acetate (2×40 ml) and washed with water (2×30 ml). The organic phase was dried over anhydrous sodium sulphate, filtered and concentrated in vacuum. (E)-3,7-dimethylocta-2,6-dienoic acid (0.40 g, 72%) was obtained by flash column chromatography (20% acetyl acetate in hexanes).

The spectral data for NQ 2984 are as follows: ¹H NMR (500 MHz, CDCl₃) δ (ppm) 1.65 (s, 3H), 1.72 (s, 3H), 2.22 (m, 7H), 5.10 (m, 1H), 5.73 (d, J=0.8 Hz, 1H); ¹³C NMR (125 MHz, CDCl₃): δ (ppm) 18.2, 19.6, 26.1, 26.5, 41.7, 115.6, 123.3, 133.2, 163.5, 172.5.

NQ 2986:

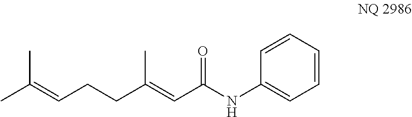 [SMILES: CC(C)=CCC/C(C)=C/C(=O)Nc1ccccc1]

To a solution of (E)-3,7-dimethylocta-2,6-dien-1-ol (400 mg, 2.4 mmol), aniline (1.1 ml, 11.9 mL) and triethylamine (2.0 mL, 14.3 mmol) in DMF (20 ml) at room temperature was added HATU (0.9 g, 2.4 mmol). The reaction was stirred overnight and quenched with water (10 mL), followed by extraction with ethyl acetate (2×20 ml). The organic phase was washed with HCl (1M, 3×20 ml) and dried over anhydrous sodium sulfate before evaporation. (E)-3,7-dimethyl-N-phenylocta-2,6-dienamide (0.36 g, 62%) was obtained by flash column chromatography (17% acetyl acetate in hexanes).

The spectral data for NQ 2986 are as follows: ¹H NMR (500 MHz, CDCl₃) δ (ppm) 1.65 (s, 3H), 1.72 (s, 3H), 2.25 (m, 7H), 5.13 (t, J=5.3 Hz, 1H), 5.73 (s, 1H), 7.12 (t, J=7.3 Hz, 1H), 7.17 (s, br, 1H), 7.36 (m, 2H), 7.58 (d, J=7.3 Hz, 2H); ¹³C NMR (125 MHz, CDCl₃): δ (ppm) 18.2, 19.0, 26.2, 26.6, 41.5, 118.6, 120.1, 123.5, 124.3, 124.4, 129.4, 132.9, 138.7, 157.3.

NQ 3052:

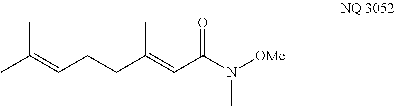

To a solution of 0.5 g (3.0 mmol) NQ 2984 in 20 ml DMF was added 1.3 g (3.0 mmol) HATU, 1.25 ml (8.9 mmol) triethylamine, and 0.44 g (4.5 mmol) N,O-Dimethylhydroxylamine hydrochloride after 5 min. The reaction was stirred overnight before quenching with water. The mixture was extracted with ethyl acetate (2×20 ml), and washed with saline three times. All solvents were removed after drying over anhydrous sodium sulfate. (E)-N-methoxy-N,3,7-trimethylocta-2,6-dienamide NQ 3052 (0.4 g, 64%) was obtained by flash column chromatography (20% ethyl acetate in hexanes).

The spectral data are as follows: ¹H NMR (700 MHz, CDCl₃) δ (ppm) 1.28 (s, 3H), 1.64 (s, 3H), 2.14 (s, 3H), 2.20 (m, 4H), 3.21 (s, 3H), 3.69 (s, 3H), 5.11 (s, 1H), 6.13 (s, br, 1H).

NQ 3055:

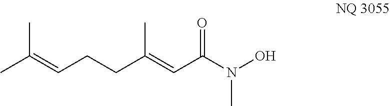

To a solution of 0.5 g (3.0 mmol) NQ 2984 in 20 ml DMF added 1.3 g (3.0 mmol) HATU, 1.25 ml (8.9 mmol) triethylamine, and 0.37 g (4.5 mmol) N-methylhydroxylamine hydrochloride after 5 min. The reaction was stirred overnight before quenching with water. The mixture was extracted with ethyl acetate (2×20 ml), and washed with saline three times. All solvents were removed after drying over anhydrous sodium sulfate. (E)-N-hydroxy-N,3,7-trimethylocta-2,6-dienamide (NQ 3055) (0.4 g, 68%) was obtained by flash column chromatography (80% ethyl acetate in hexanes).

The spectral data are as follows: ¹H NMR (500 MHz, CDCl₃) δ (ppm) 1.66 (s, 3H), 1.73 (s, 3H), 2.07 (s, 3H), 2.22 (m, 4H), 3.34 (s, 3H), 5.12 (s, 1H), 5.77 (s, br, 1H), 8.77 (s, br, 1H).

### Example 9

The following examples were synthesised according to Scheme 7:

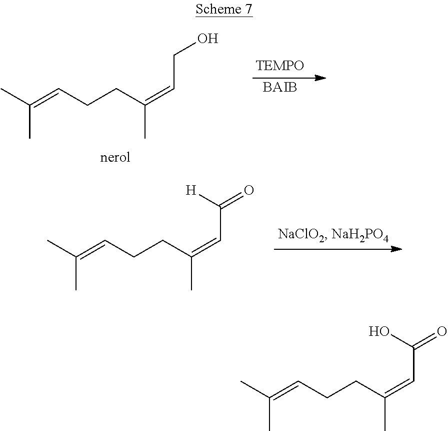

NQ 3000:

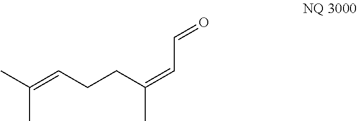

To a solution of 3.0 g (19.5 mmol) nerol in 15 ml dichloromethane added 6.3 g (19.5 mmol) BAIB and 0.3 g (1.9 mmol) TEMPO. The reaction was stirred for 2 hour before quenching with 20 ml saturated sodium thiosulfate. The mixture extracted with ethyl acetate (3×40 ml), and dried over anhydrous sodium sulfate. (Z)-3,7-dimethylocta-2,6-dienal (NQ 3000) (2.5 g, 84%) was obtained by flash column chromatography (10% ethyl acetate in hexanes).

The spectral data are as follows: ¹H NMR (500 MHz, CDCl₃) δ (ppm) 1.66 (s, 3H), 1.74 (s, 3H), 2.02 (s, 3H), 2.30 (q, J=7.4 Hz, 2H), 2.65 (t, J=7.4 Hz, 2H), 5.16 (t, J=7.3 Hz, 1H), 5.94 (d, J=8.1 Hz, 1H), 9.95 (d, J=8.1 Hz, 1H); ¹³C NMR (125 MHz, CDCl₃): δ (ppm) 17.3, 24.7, 25.2, 26.6, 32.2, 121.8, 128.2, 133.2, 163.4, 190.4.

NQ 3001:

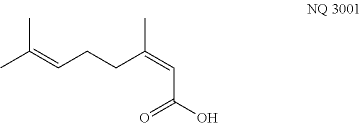

To a solution of 1.5 g (9.8 mmol) (Z)-3,7-dimethylocta-2,6-dienal in 20 ml DMSO added 10.4 ml (99 mmol) 2-methyl-2-butene, and slowly added 8.9 g (99 mmol) sodium chlorite and 8.3 g (69 mmol) sodium chlorite in 20 ml water. The reaction was stirred for 2 hours before quenching with 20 ml saturated sodium thiosulfate. The mixture was extracted with ethyl acetate (2×20 ml) and dried over anhydrous sodium sulfate. (Z)-3,7-dimethylocta-2,6-dienoic acid NQ 3001 (0.50 g, 30%) was obtained by flash column chromatography (20% acetyl acetate in hexanes).

The spectral data are as follows: ¹H NMR (500 MHz, CDCl₃) δ (ppm) 1.65 (s, 3H), 1.72 (s, 3H), 1.96 (s, 3H), 2.21 (m, 2H), 2.68 (t, J=8.2 Hz, 2H), 5.17 (m, 1H), 5.72 (d, J=0.9 Hz, 1H); ¹³C NMR (125 MHz, CDCl₃): δ (ppm) 17.7, 25.8, 25.9, 27.0, 33.9, 115.9, 123.7, 132.6, 163.7, 171.8.

### Example 10

The following examples were synthesised according to Scheme 8:

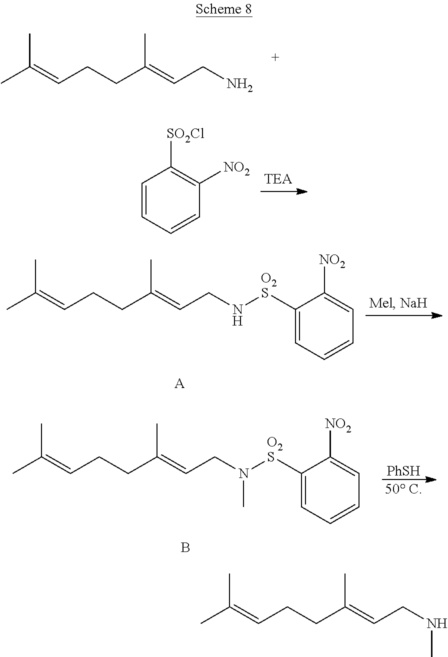

NQ 2991:

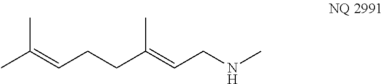

To a solution of 0.5 (3.3 mmol) geranylamine and 0.7 ml (4.9 mmol) triethylamine in 20 ml dichloromethane slowly added 0.72 g (6.9 mmol) 2-nitrobenzene-1-sulfonyl chloride cooling in ice water. The reaction was stirred for 1 hour before quenching with 50 ml water. The mixture was extracted with ethyl acetate (2×20 ml), and dried over anhydrous sodium sulfate. Compound A (1.1 g, 100%) was obtained by a flash column chromatography (50% ethyl acetate in hexanes).

Compound A (1.1 g, 3.3 mmol) was added to a suspension of 0.1 g (3.9 mmol) sodium hydride in 20 ml anhydrous THF cooling in ice water, followed by the addition of 0.24 ml (3.9 mmol) iodomethane after half an hour. The reaction was stirred overnight before quenching with water. The reaction mixture was extracted with ethyl acetate (2×20 ml) and dried over anhydrous sodium sulfate. Compound B (1.0 g, 90%) was obtained by a flash column chromatography (30% ethyl acetate in hexanes).

To a solution of 0.72 g (6.8 mmol) thiophenol in 30 ml acetonitrile was added 0.39 g (6.9 mmol) potassium hydroxide in 10 ml water under argon atmosphere, cooling in ice water. The mixture was stirred for 10 min before adding 1.1 g (3.1 mmol) compound B. The reaction was stirred for 2 hours at 50′C before quenching with 100 ml water. The mixture was extracted with ethyl acetate (2×20 ml), and dried over anhydrous sodium sulfate. Compound 2991 (0.30 g, 58%) was obtained by a flash column chromatography (150 ml acetone first, followed by 200 ml methanol).

The spectral data are as follows: ¹H NMR (500 MHz, CDCl₃) δ (ppm) 1.27 (s, 3H), 1.61 (s, 3H), 1.66 (s, 3H), 2.10 (m, 4H), 2.59 (s, 3H), 3.58 (d, J=7.1 Hz, 2H), 5.07 (m, 1H), 5.39 (t, J=8.5 Hz, 1H), 7.33 (s, 1H); ¹³C NMR (125 MHz, CDCl₃): δ (ppm) 17.0, 18.1, 26.1, 26.6, 31.8, 40.1, 46.4, 114.8, 123.8, 132.6, 146.2.

### Example 11

The following examples were synthesised according to Scheme 9:

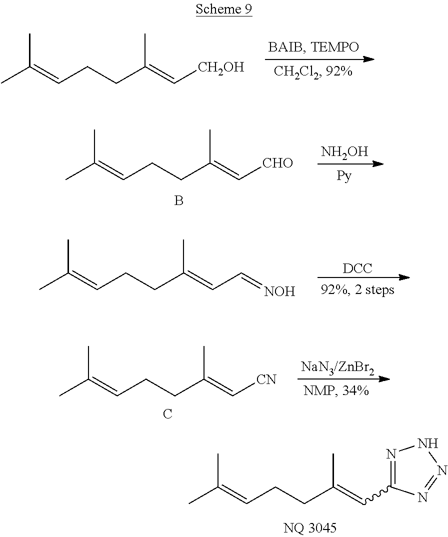

NQ 3045:

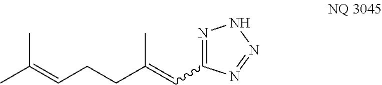

Geraniol (3.086 g, 20 mmol), BAIB (6.44 g, 20 mmol) and TEMPO (313 mg, 2 mmol) were stirred in CH₂Cl₂ (50 mL) at room temperature for 3 h. The solution was washed with saturated aqueous Na₂S₂O₃, saturated NaHCO₃ and brine. The organic layer was dried with Na₂SO₄, and concentrated. The residue was purified with flash chromatography to afford B (2.8 g, 92%) as a colourless oil.

Hydroxylamine hydrochloride (584 mg, 8.4 mmol) and compound B (1.216 g, 8 mmol) were stirred at room temperature in pyridine/H₂O (4 mL, 1:1) for 1 hour. Then copper sulfate (256 mg, 1.6 mmol) and triethylamine (1.7 g, 16.8 mmol) in CH₂Cl₂ (8 mL) were added to the mixture. After stirring for 10 min, DCC in CH₂Cl₂ (16 mL) was added, and the mixture was stirred for 4 hours. The reaction was quenched with 1 N HCl, and the mixture was extracted with CH₂Cl₂. The organic layer was washed with saturated NaHCO₃ and brine. The solution was dried with Na₂SO₄, and concentrated. The residue was purified with flash chromatography to afford C (1.1 g, 92%) as a colorless oil. ¹H NMR (700 MHz, CDCl₃) δ (ppm) 1.60 (s, 3H), 1.68 (s, 3H), 2.04 (s, 3H), 2.14 (m, 2H), 2.20 (t, 2H), 5.01 (t, 1H), 5.10 (s, 1H); ¹³C NMR (175 MHz, CDCl₃): 17.75, 21.08, 25.60, 25.68, 38.59, 95.23, 117.35, 122.16, 133.26, 165.08.

Compound C (675 mg, 4.53 mmol), sodium azide (1.176 g, 18.1 mmol) and zinc bromide (4.07 g, 18.1 mmol) in NMP (15 mL) were heated at 170° C. overnight under argon atmosphere. After cooling to ambient temperature, the mixture was diluted with EtOAc and 1 N HCl, and the mixture was washed with brine. The organic layers were dried over Na₂SO₄, filtered and evaporated. The residue was purified by flash chromatography to afford NQ 3045 (Z/E mixture) (300 mg, 34% yield).

The spectral data for the NQ 3045 Z isomer are as follows: ¹H NMR (700 MHz, CDCl₃) δ (ppm) 1.59 (s, 3H), 1.64 (s, 3H), 2.02 (s, 3H), 2.22 (m, 2H), 2.69 (t, 2H), 5.13 (t, 1H), 6.39 (s, 1H); ¹³C NMR (175 MHz, CDCl₃): 17.73, 19.77, 24.94, 26.02, 34.25, 106.70, 123.32, 133.24, 153.16, 154.19.

The spectral data for the NQ 3045 E isomer are as follows: ¹H NMR (700 MHz, CDCl₃) δ (ppm) 1.59 (s, 3H), 1.66 (s, 3H), 2.22 (m, 2H), 2.26 (s, 3H), 2.30 (t, 2H), 5.09 (t, 1H), 6.41 (s, 1H); ¹³C NMR (175 MHz, CDCl₃): 17.73, 19.77, 25.69, 26.17, 40.81, 105.98, 122.83, 132.74, 153.49, 154.11.

### Example 12

The following examples were synthesised according to Scheme 10:

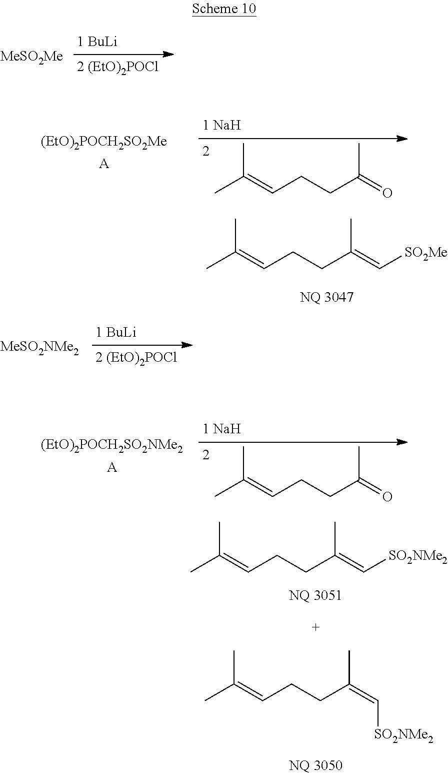

NQ 3047:

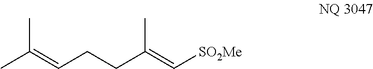

Under argon atmosphere n-BuLi 2.0 M in hexanes (3.6 mL, 7.2 mmol) was added to a solution of methylsulfonylmethane (564 mg, 6 mmol) in THF (30 mL) cooled at −78° C. The resulting solution was stirred at 0° C. for 30 min, and then brought back to −78° C. Diethyl chlorophosphate (0.72 mL, 5 mmol) was added, and the temperature allowed to slowly raise room temperature and stirred for 3 hours. Then, NaH (252 mg, 10 mmol) was added. After stirring for 1 hour at room temperature, 6-methylhept-5-en-2-one (0.74 mL, 5 mmol) was added to the solution, and the mixture was stirred overnight. Then, a saturated aqueous solution of NH₄Cl (30 mL) was added, the organic layer was separated and the aqueous layer was extracted with CH₂Cl₂ (3×15 mL). The combined organic layers were dried (Na₂SO₄) and the solvent was evaporated. The residue was purified by flash chromatography chromatography to afford NQ 3047 (343 g, 34%) as a colorless oil.

The spectral data are as follows: ¹H NMR (700 MHz, CDCl₃) δ (ppm) 1.62 (s, 3H), 1.70 (s, 3H), 2.18-2.21 (m, 7H), 2.95 (s, 3H), 5.05-5.06 (m, 1H), 6.12 (s, 1H); ¹³C NMR (175 MHz, CDCl₃): 17.17, 17.89, 25.61, 25.69, 40.25, 43.80, 122.08, 125.21, 133.34, 158.28.

NQ 3050 and NQ 3051

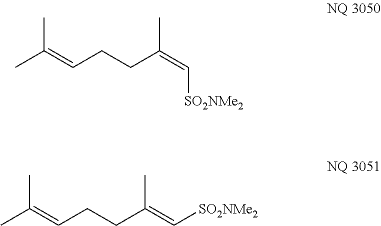

Under argon atmosphere n-BuLi 2.0 M in hexanes (4.8 mL, 9.6 mmol) was added to a solution of N,N-dimethylmethanesulfonamide (984 mg, 8 mmol) in THF (40 mL) cooled at −78° C. The resulting solution was stirred at 0° C. for 30 min, and then brought back to −78° C. Diphenylphosphinic chloride (1.5 mL, 8 mmol) was added, and the temperature allowed to slowly raise room temperature and stirred for 3 hours. Then, a saturated aqueous solution of NH₄Cl (30 mL) was added, the organic layer was separated and the aqueous layer was extracted with CH₂Cl₂ (3×15 mL). The combined organic layers were dried (Na₂SO₄) and the solvent was evaporated. The residue was purified by flash chromatography to afford B (1.2 g, 46.4%) as a white solid.

The spectral data are as follows: ¹H NMR (700 MHz, CDCl₃) δ (ppm) 2.92 (s, 6H), 4.09 (d, 2H), 7.52-7.55 (m, 4H), 7.59-7.61 (m, 2H), 7.83-7.86 (m, 4H); ¹³C NMR (175 MHz, CDCl₃): 37.46, 50.66, 51.00, 128.77, 128.84, 130.88, 131.06, 131.12, 131.48, 132.52, 132.54.

The same method outlined for NQ 3047, OMB 3050 and NQ 3051 was afforded with N,N-dimethylmethanesulfonamide as the starting material instead.

¹H NMR data for NQ 3051: ¹H NMR (500 MHz, CDCl₃) δ (ppm) 1.62 (s, 3H), 1.69 (s, 3H), 2.12 (s, 3H), 2.13-2.23 (m, 4H), 2.76 (s, 6H), 5.04-5.05 (m, 1H), 5.86 (d, J=1.1, 1H); ¹³C NMR (125 MHz, CDCl₃): 17.88, 18.03, 25.82, 25.89, 37.58, 40.69, 118.16, 122.56, 133.15, 156.79.

¹H NMR data for NQ 3050: ¹H NMR (500 MHz, CDCl₃) δ (ppm) 1.68 (s, 3H), 1.73 (s, 3H), 2.00 (d, J=1.8, 3H), 2.21-2.26 (m, 2H), 2.63-2.66 (m, 2H), 283 (s, 6H), 5.19 (t, J=8.2, 1H), 5.91 (s, 1H); ¹³C NMR (125 MHz, CDCl₃): 17.71, 24.82, 25.73, 26.87, 32.60, 37.58, 118.36, 123.04, 132.77, 157.12.

### Example 13

The following examples were synthesised according to Scheme 11:

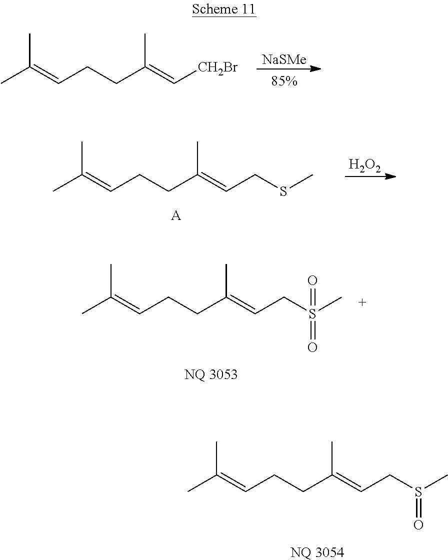

NQ3053 and NQ3054:

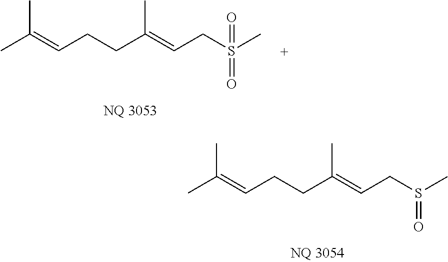

Under argon atmosphere geranyl bromide (0.76 mL, 4 mmol) was added to sodium thiomethoxide (280 mg, 4 mmol) in dichloromethane solution (15 mL) at −20° C. The resulting mixture was stirred for 3 hours at −20° C. and slowly warmed to room temperature. Then, brine was added, the organic layer was separated and the aqueous layer was extracted with CH₂Cl₂. The combined organic layers were dried (Na₂SO₄) and the solvent was evaporated. The residue was purified by flash chromatography to afford compound A (626 mg, 85%) as a colorless oil.

The spectral data are as follows: ¹H NMR (500 MHz, CDCl₃) δ (ppm): 1.77 (s, 3H), 1.83 (s, 3H), 1.86 (s, 3H), 2.21 (s, 3H), 2.23-2.28 (m, 4H), 3.30-3.32 (m, 2H), 5.27 (s, 1H), 5.43 (m, 1H); ¹³C NMR (125 MHz, CDCl₃): 14.46, 16.21, 17.88, 25.89, 26.67, 31.25, 39.81, 120.41, 124.14, 131.84, 139.02.

Hydrogen peroxide (30% in H₂O, 1.36 mL, 13.37 mmol) was added to compound 4 (1.64 g, 8.91 mmol) in methol (20 mL) at −10° C. The resulting mixture was stirred for 2 hours at −10° C. and slowly warmed to room temperature. Then, the mixture was concentrated under vacuum, and the residue was purified by flash chromatography. NQ 3054 is the major product, and NQ 3053 is the minor product.

The spectral data for NQ 3053 are as follows: ¹H NMR (500 MHz, CDCl₃) δ (ppm): 1.65 (s, 3H), 1.73 (s, 3H), 1.79 (s, 3H), 2.20 (d, J=2.9, 4H), 2.86 (s, 3H), 3.78 (d, J=7.9, 1H), 5.10 (s, 1H), 5.40 (t, J=7.9, 1H); ¹³C NMR (125 MHz, CDCl₃): 16.71, 17.77, 25.78, 26.09, 38.87, 39.68, 54.72, 110.93, 123.34, 132.34, 146.19.

The spectral data for NQ 3054 are as follows: ¹H NMR (500 MHz, CDCl₃) δ (ppm) 1.59 (s, 3H), 1.66 (s, 3H), 1.72 (s, 3H), 2.10 (s, 4H), 2.51 (s, 3H),), 3.39-3.44 (m, 1H), 3.54-3.58 (m, 1H), 5.03 (s, 1H), 5.23 (t, J=7.8, 1H); ¹³C NMR (125 MHz, CDCl₃): 16.86, 17.72, 25.73, 26.20, 37.08, 39.70, 53.41, 110.98, 123.51, 132.02, 145.27.

### Example 14

The following examples were synthesised according to Scheme 12:

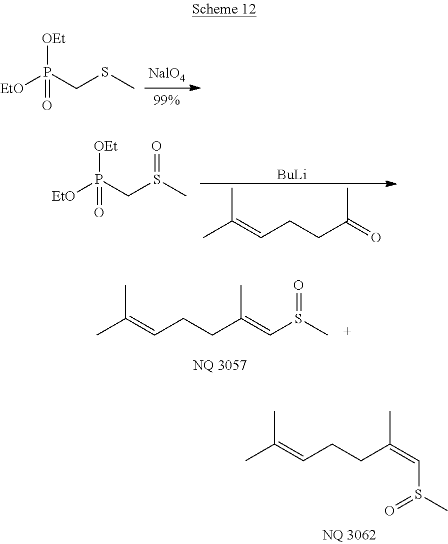

NQ 3057 and NQ 3062:

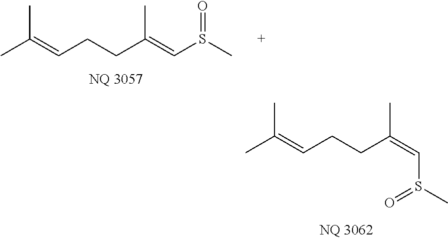

A solution of sodium metaperiodate (1.91 g, 8.9 mmol) in water (25 mL) was dropwise added to a solution of diethyl(methylthiomethyl)phosphonate (1.69 g, 8.5 mmol) in acetone (6 mL) at 0° C. The mixture was stirred for 4 h and concentrated under vacuum. The residue was extracted with CH₂Cl₂. The organic layer were dried (Na₂SO₄) and the solvent was evaporated. The residue was purified by flash chromatography to afford its sulphoxide as colourless oil.

The spectral data are as follows: ¹H NMR (500 MHz, CDCl₃) δ (ppm) 1.41 (t, J=7.0, 6H), 2.90 (s, 3H), 3.30-3.43 (m, 2H), 4.20-4.27 (m, 4H; ¹³C NMR (125 MHz, CDCl₃): 16.37, 16.42, 41.26, 41.29, 50.89, 51.97, 62.98, 63.00, 63.03, 63.05.

Under argon atmosphere n-BuLi 2.0 M in hexanes (5 mL, 10 mmol) was added to a solution of phosphoryl sulphoxide (1.78 g, 8.32 mmol) in THF (25 mL) cooled at −78° C. The resulting solution was stirred at −78° C. for 20 min, and then 6-methylhept-5-en-2-one (1.23 mL, 8.32 mmol) was added to the solution. The mixture was stirred overnight at room temperature. The reaction was quenched by saturated aqueous solution of NH₄Cl, the organic layer was separated and the aqueous layer was extracted with EtOAc. The combined organic layers were dried (Na₂SO₄) and the solvent was evaporated. The residue was purified by flash chromatography to afford NQ 3057 and NQ 3062.

The spectral data for NQ 3057 are as follows: ¹H NMR (500 MHz, CDCl₃) δ (ppm): 1.63 (s, 3H), 1.72 (s, 3H), 1.94 (s, 3H), 2.12-2.18 (m, 1H), 2.21-2.27 (m, 1H), 2.32-2.38 (m, 1H), 2.55-2.63 (m, 1H), 2.61 (s, 3H), 5.06-5.09 (m, 1H), 6.11 (d, J=1.0, 1H); ¹³C NMR (125 MHz, CDCl₃): 17.74, 23.25, 25.76, 26.44, 33.99, 40.35, 122.58, 131.99, 133.31, 151.96.

The spectral data for NQ 3062 are as follows: ¹H NMR (500 MHz, CDCl₃) δ (ppm): 1.65 (s, 3H), 1.73 (s, 3H), 1.04 (s, 3H), 2.21-2.22 (m, 4H), 2.64 (s, 3H), 5.10 (s, br, 1H), 6.11 (d, J=1.0, 1H); ¹³C NMR (125 MHz, CDCl₃): 17.79, 18.71, 25.64, 25.71, 39.06, 40.26, 122.61, 130.94, 132.91, 152.08.

### Example 15

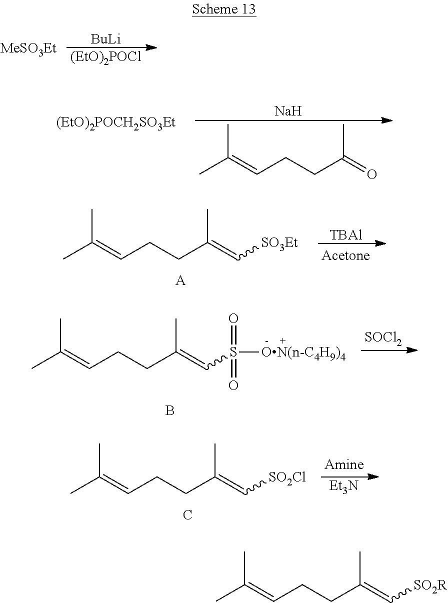

Under argon atmosphere n-BuLi 2.0 M in hexanes (16.5 mL, 33 mmol) was added to a solution of ethyl methanesulfonate (3.72 mg, 30 mmol) in THF (60 mL) cooled at −78° C. The resulting solution was stirred at −78° C. for 30 min, and then diethyl chlorophosphate (3.61 mL, 25 mmol) was added. The temperature was allowed to slowly raise room temperature and stirred for 1 hour. Then, NaH (1.2 g, 50 mmol) was added. After stirring for 1 hour at room temperature, 6-methylhept-5-en-2-one (3.7 mL, 25 mmol) was added to the solution, and the mixture was stirred overnight. The reaction was quenched by saturated aqueous solution of NH₄Cl, the organic layer was separated and the aqueous layer was extracted with CH₂Cl₂. The combined organic layers were dried (Na₂SO₄) and the solvent was evaporated. The residue was purified by flash chromatography to afford A (2.7 g, 46.6%) as a colorless oil (Z/E mixture).

The spectral data are as follows: ¹H NMR (500 MHz, CDCl₃) δ (ppm) 1.56 (m, 3H), 1.79 (s, 3H), 1.87 (s, 3H), 2.32 (s, 3H), 2.33-2.40 (m, 4H), 4.36 (m, 2H), 5.22-5.31 (m, 1H), 6.23 (s, 1H); ¹³C NMR (125 MHz, CDCl₃): 15.08, 17.93, 18.53, 25.71, 25.85, 40.24, 66.14, 120.39, 122.22, 133.56, 159.23.

Vinyl sulfonate ester A (2.13 g, 9.18 mmol) was dissolved in 25 mL anhydrous acetone, and then Bu₄NI (3.38 g, 9.18 mmol) was added. The resulting mixture was stirred at reflux for 3 days. The acetone was removed by rotary evaporation under vacuum to afford the crude vinyl sulfonate tetrabutylammonium salt B, which was used without further purification. The crude vinyl sulfonate tetrabutylammonium salt B (1 g, 2.26 mmol) was dissolved in 10 mL CH₂Cl₂ and cooled to 0° C. PPh₃ (1.57 mg, 6 mmol) and SOCl₂ (0.44 mL, 6 mol) were added. The resulting reaction mixture was stirred at 0° C. for 1 hour, then warmed to rt and stirring was continued for 2 h. The mixture was concentrated by rotary evaporation and purified by flash chromatography to afford C (Z/E mixture, 1:3).

The spectral data are as follows: ¹H NMR (500 MHz, CDCl₃) δ (ppm) 1.66 (s, 3H), 1.75 (s, 3H), 2.25-2.35 (m, 7H), 5.07 (t, 1H), 6.61 (s, 1H); ¹³C NMR (125 MHz, CDCl₃): 17.82, 18.84, 25.48, 25.73, 40.04, 121.42, 129.50, 134.07, 162.18.

The following examples were synthesised according to Scheme 13:

NQ 3060:

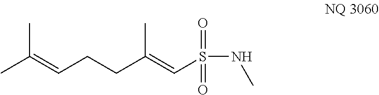

Under argon atmosphere methylamine (8 M in EtOH, 1 mL, 1 mmol) was added to a solution of (E)-2,6-dimethylhepta-1,5-diene-1-sulfonyl chloride (240 mg, 1.08 mmol) and triethylamine (0.15 mL, 1.08 mmol) in CH₂Cl₂ at 0° C. The resulting mixture was stirred for 10 min at 0° C., and then brine was added. The organic layer was separated and the aqueous layer was extracted with CH₂Cl₂. The combined organic layers were dried (Na₂SO₄) and the solvent was evaporated. The residue was purified by flash chromatography to afford compound NQ 3060 (220 mg, 94%) as a colorless oil.

The spectral data are as follows: ¹H NMR (500 MHz, CDCl₃) δ (ppm): 1.65 (s, 3H), 1.73 (s, 3H), 2.16 (d, J=1.1, 3H), 2.22-2.24 (m, 4H), 2.76 (d, J=5.2, 3H), 4.46 (d, br, J=5.0, 1H), 5.08-5.10 (m, 1H), 6.01 (s, 1H); ¹³C NMR (125 MHz, CDCl₃): 17.29, 17.30, 25.21, 25.23, 28.44, 39.81, 121.47, 121.78, 132.67, 155.61.

NQ3061:

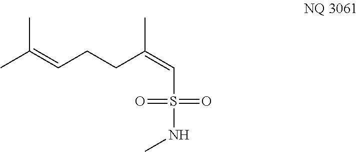

NQ 3061 was synthesized using the same method as above using (4-2,6-dimethylhepta-1,5-diene-1-sulfonyl chloride.

The spectral data are as follows: ¹H NMR (500 MHz, CDCl₃) δ (ppm): 1.61 (s, 3H), 1.67 (s, 3H), 1.92 (d, J=1.1, 3H), 2.16-2.20 (m, 2H), 2.55-2.58 (m, 2H), 2.72 (d, J=5.4, 3H), 4.37 (s, br, 1H), 5.12 (t, J=7.2, 1H), 5.98 (s, 1H); ¹³C NMR (125 MHz, CDCl₃): 17.74, 24.53, 25.78, 26.59, 29.12, 32.44, 122.52, 123.00, 132.94, 156.41.

NQ 3063:

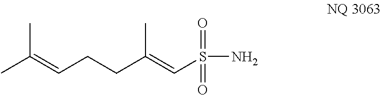

Ammonium hydroxide solution (30% in water, 2 mL) was added to a solution of (E)-2,6-dimethylhepta-1,5-diene-1-sulfonyl chloride (280 mg, 1.26 mmol) in THF at room temperature. The resulting mixture was stirred for 1 h, and then brine was added. The organic layer was separated and the aqueous layer was extracted with CH₂Cl₂. The combined organic layers were dried (Na₂SO₄) and the solvent was evaporated. The residue was purified by flash chromatography to afford compound NQ 3063 (200 mg, 78%) as a colorless oil.

The spectral data are as follows: ¹H NMR (500 MHz, CDCl₃) δ (ppm): 1.66 (s, 3H), 1.68 (s, 3H), 2.17 (s, 3H), 2.21-2.22 (m, 4H), 4.85 (Br, 2H), 5.10 (br, 1H), 6.28 (s, 1H); ¹³C NMR (125 MHz, CDCl₃): 17.81, 18.00, 25.67, 25.76, 40.05, 122.25, 125.81, 133.27, 154.71.

NQ3064:

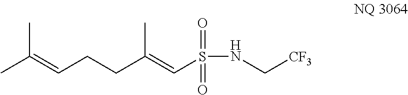 [SMILES: CC(C)=CCC/C(C)=C/S(=O)(=O)NCC(F)(F)F]

(E)-2,6-dimethylhepta-1,5-diene-1-sulfonyl chloride (230 mg, 1.03 mmol) was added to a solution of 2,2,2-trifluoroethylamine hydrochloride (500 mg, 3.69 mmol) and triethylamine (1 mL, 7.18 mmol) in methanol at room temperature. The resulting mixture was stirred for 2 h, and then brine was added. The organic layer was separated and the aqueous layer was extracted with CH₂Cl₂. The combined organic layers were dried (Na₂SO₄) and the solvent was evaporated. The residue was purified by flash chromatography to afford compound NQ 3064 (200 mg, 51%) as a white solid.

The spectral data are as follows: ¹H NMR (500 MHz, CDCl₃) δ (ppm): 1.66 (s, 3H), 1.74 (s, 3H), 2.16 (s, 3H), 2.21-2.25 (m, 4H), 3.71-3.78 (m, 2H), 5.05-5.10 (m, 2H), 6.13 (s, 1H); ¹³C NMR (125 MHz, CDCl₃): 17.76, 17.95, 25.61, 25.69, 40.24, 43.90, 44.18, 44.46, 44.74, 122.12, 122.61, 123.44, 124.82, 127.04, 133.35, 156.63.

NQ 3069:

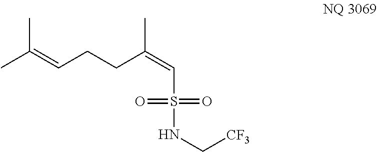 [SMILES: CC(C)=CCC/C(C)=C\S(=O)(=O)NCC(F)(F)F]

NQ 3069 was afforded using the same method as NQ 3064 but using (Z)-2,6-dimethylhepta-1,5-diene-1-sulfonyl chloride.

The spectral data are as follows: ¹H NMR (500 MHz, CDCl₃) δ (ppm): 1.68 (s, 3H), 1.74 (s, 3H), 1.98 (d, J=0.5, 3H), 2.23-2.26 (m, 2H), 2.61-2.64 (m, 2H), 3.74-3.77 (m, 2H), 4.96 (br, 1H), 5.18 (br, 1H), 6.12 (s, 1H); ¹³C NMR (125 MHz, CDCl₃): 17.69, 24.47, 25.71, 26.42, 29.75, 32.51, 43.94, 44.22, 44.50, 44.78, 122.61, 122.78, 123.86, 124.83, 133.19, 156.69.

NQ 3070:

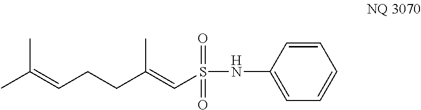 [SMILES: CC(C)=CCC/C(C)=C/S(=O)(=O)Nc1ccccc1]

(E)-2,6-dimethylhepta-1,5-diene-1-sulfonyl chloride (295 mg, 1.32 mmol) was added to a solution of benzyl amine (0.3 ml, 2.86 mmol) and triethylamine (0.3 ml, 2.16 mmol) in CH₂Cl₂ at room temperature. The resulting mixture was stirred for 1 h, and then brine was added. The organic layer was separated and the aqueous layer was extracted with CH₂Cl₂. The combined organic layers were dried (Na₂SO₄) and the solvent was evaporated. The residue was purified by flash chromatography to afford compound NQ 3070 (350 mg, 90%) as a colorless oil.

The spectral data are as follows: ¹H NMR (500 MHz, CDCl₃) δ (ppm): 1.66 (s, 3H), 1.74 (s, 3H), 2.12 (d, J=1.1, 3H), 2.13-2.15 (m, 4H), 4.24 (d, 2H), 4.96 (t, J=6.3, 1H), 5.08 (br, 1H), 6.01 (s, 1H), 7.32-7.40 (m, 5H); ¹³C NMR (125 MHz, CDCl₃): 17.83, 17.92, 25.67, 25.77, 40.20, 46.92, 122.40, 123.56, 127.92, 127.97, 128.75, 133.11, 136.88, 155.42.

NQ3071:

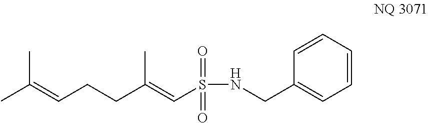 [SMILES: CC(C)=CCC/C(C)=C/S(=O)(=O)NCc1ccccc1]

(E)-2,6-dimethylhepta-1,5-diene-1-sulfonyl chloride (340 mg, 1.53 mmol) was added to a solution of phenyl amine (0.91 ml, 15.3 mmol) in CH₂Cl₂ at room temperature. The resulting mixture was stirred for 4 h, and then dilute HCl was added. The organic layer was separated and the aqueous layer was extracted with CH₂Cl₂. The combined organic layers were washed with brine and dried (Na₂SO₄) and the solvent was evaporated. The residue was purified by flash chromatography to afford compound NQ 3071 (410 mg, 96%) as a colorless oil.

The spectral data are as follows: ¹H NMR (500 MHz, CDCl₃) δ (ppm): 1.59 (s, 3H), 1.67 (s, 3H), 2.05 (s, 3H), 2.09-2.13 (m, 2H), 2.14-2.17 (m, 2H), 4.95-4.8-98 (m, 1H), 6.14 (s, 1H), 7.16 (s, 1H), 7.19 (t, J=7.4, 1H), 7.24 (d, J=7.9, 2H), 7.36 (t, J₁=7.6, J₂=8.0, 2H); ¹³C NMR (125 MHz, CDCl₃): 17.73, 17.98, 25.65, 25.66, 40.25, 121.03, 122.12, 122.79, 124.99, 129.37, 133.16, 136.95, 157.27.

### Example 16

The following examples were synthesised according to Scheme 14:

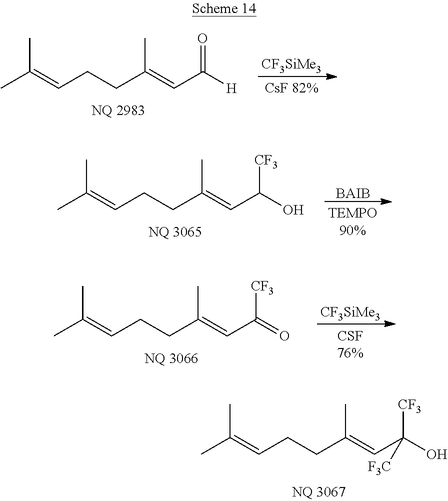

NQ 3065:

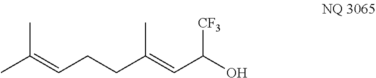

To a solution of 0.5 g (3.3 mmol) NQ 2983 in 20 ml anhydrous THF was added 0.53 ml (3.6 mmol) trifluoromethyltrimethylsilane, 0.1 g (0.66 mmol) cesium fluoride under argon atmosphere. The reaction was stirred overnight before quenching with water and 10 ml 6N HCl. The mixture was extracted with ethyl acetate (2×20 ml). All solvents were removed after drying over anhydrous sodium sulfate. (E)-1,1,1-trifluoro-4,8-dimethylnona-3,7-dien-2-ol NQ 3065 (0.6 g, 82%) was obtained by flash column chromatography (10% ethyl acetate in hexanes).

The spectral data are as follows: ¹H NMR (500 MHz, CDCl₃) δ (ppm) 1.65 (s, 3H), 1.73 (s, 3H), 1.85 (s, 3H), 2.08 (d, J=7.6 Hz, 1H), 2.16 (m, 4H), 4.73 (m, 1H), 5.11 (m, 1H), 5.32 (d, J=8.7 Hz, 1H). ¹³C NMR (125 MHz, CDCl₃): δ (ppm) 17.1, 17.7, 25.7, 26.0, 39.6, 67.8 (q, J=32.2 Hz), 117.0 (q, J=1.7 Hz), 123.2, 126.0 (q, J=282.0 Hz), 132.4, 146.5.

NQ3066:

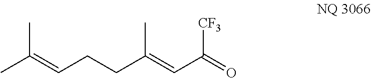

To a solution of 0.5 g (2.3 mmol) (E)-1,1,1-trifluoro-4,8-dimethylnona-3,7-dien-2-ol 10 ml dichloromethane add 0.73 g (2.3 mmol) iodobenzen diacetate and 0.035 g (0.2 mmol) TEMPO, stirring for 4 hours at room temperature. The reaction was quenched with 10 ml saturated sodium thiosulfate solution and the mixture was extracted with ethyl acetate (3×20 ml). Solvents were removed under vacuum after drying over anhydrous sodium sulfate. (E)-1,1,1-trifluoro-4,8-dimethylnona-3,7-dien-2-one (NQ 3066) (0.5 g, 100%) was obtained by flash column chromatography (6% ethyl acetate in hexanes). This final product was purified again by flash column (10% dichloromethane in hexanes) when proton NMR showed unidentified peaks at lower field.

The spectral data are as follows: ¹H NMR (500 MHz, CDCl₃) δ (ppm) 1.66 (s, 3H), 1.74 (s, 3H), 2.28 (m, 2H), 2.35 (m, 5H), 5.11 (m, 1H), 6.36 (m, 1H). ¹³C NMR (125 MHz, CDCl₃): δ (ppm) 17.8, 21.2, 25.7, 25.9, 42.1, 115.0, 117.4, 122.2, 133.4, 172.0, 179.8.

NQ 3067:

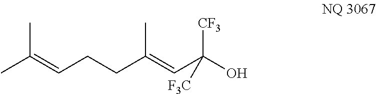 [SMILES: CC(C)=CCC/C(C)=C/C(O)(C(F)(F)F)C(F)(F)F]

To a solution of 0.5 g (2.3 mmol) (E)-1,1,1-trifluoro-4,8-dimethylnona-3,7-dien-2-ol (2) in 20 ml anhydrous THF added 0.37 ml (2.5 mmol) trifluoromethyltrimethylsilane, 0.07 g (0.45 mmol) cesium fluoride under argon atmosphere. The reaction was stirred overnight before quenching with water and 10 ml 6N HCl. The mixture was extracted with ethyl acetate (2×10 ml). All solvents were removed after drying over anhydrous sodium sulfate. (E)-1,1,1-trifluoro-4,8-dimethyl-2-(trifluoromethyl)nona-3,7-dien-2-ol (NQ 3067) (0.5 g, 76%) was obtained by flash column chromatography (10% ethyl acetate in hexanes).

The spectral data are as follows: ¹H NMR (500 MHz, CDCl₃) δ (ppm) 1.66 (s, 3H), 1.73 (s, 3H), 2.10 (s, 3H), 2.18 (m, 4H), 2.91 (s, 1H), 5.09 (m, 1H), 5.28 (s, 1H). ¹³C NMR (125 MHz, CDCl₃): δ (ppm) 17.7, 17.8, 25.6, 26.1, 41.5, 111.3, 121.7, 122.8, 124.0, 132.6, 150.3.

### Example 17

The following examples were synthesised according to Scheme 15:

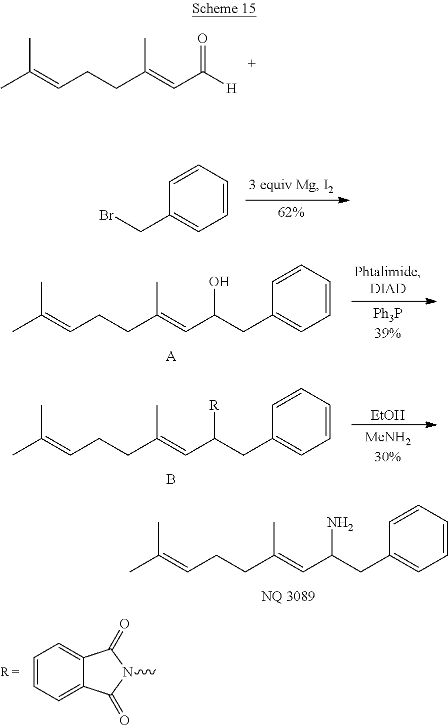

NQ 3089:

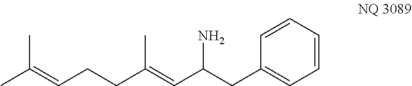 [SMILES: CC(C)=CCC/C(C)=C/C(N)Cc1ccccc1]

To a suspension of 0.5 g (19.7 mmol) magnesium and a few iodine crystals in 20 ml anhydrous ethyl ether was added 0.8 ml (6.6 mmol) benzylbromide under argon atmosphere. The reaction was stirred for half an hour while boiling, recovering to room temperature before adding 1.0 g (6.6 mmol) geranyl aldehyde. The reaction mixture was stirred overnight at room temperature. The next day, the reaction was quenched with water and 10 ml saturated ammonium chloride solution cooling in ice water. The mixture was extracted with ethyl acetate (2×20 ml) and dried over anhydrous sodium sulfate. (E)-4,8-dimethyl-1-phenylnona-3,7-dien-2-ol (A) (1.0 g, 62%) was obtained by flash column chromatography (15% ethyl acetate in hexanes).

To a solution of 1.4 g (5.3 mmol) triphenylphosphine and 0.8 g (5.3 mmol) phthalimide in 30 ml anhydrous THF was added 1.0 g compound A and 0.9 ml (5.3 mmol) diisopropyl azodicarboxylate under argon atmosphere. The reaction was stirred overnight before removing all the solvents next day. The residue was extracted with ethyl ether/hexanes (1/1, 2×15 ml). (E)-2-(4,8-dimethyl-1-phenylnona-3,7-dien-2-yl)isoindoline-1,3-dione B (0.6, 39%) obtained by flash column chromatography (10% ethyl acetate+1% ethyl ether in hexanes).

To a solution of 0.5 g (1.4 mmol) (E)-2-(4,8-dimethyl-1-phenylnona-3,7-dien-2-yl)isoindoline-1,3-dione (B) in 20 ml anhydrous ethanol was added 1.0 ml (8.4 mmol) 8 N methylamine. The reaction was refluxed for 4 hours before removing the solvents. The mixture was filtered after dissolving in dichloromethane/hexanes (1:1). NQ 3089 (0.1 g, 30%) was obtained by flash column chromatography (10% methanol+1% ethyl ether+0.5% triethylamine in dichloromethane).

The spectral data are as follows: ¹H NMR (500 MHz, CDCl₃) d (ppm) 1.55 (s, 3H), 1.63 (m, 5H), 1.73 (s, 3H), 2.07 (m, 4H), 2.68 (m, 2H), 3.86 (m, 1H), 5.12 (m, 2H), 7.24 (m, 3H), 7.31 (m, 2H). ¹³C NMR (125 MHz, CDCl₃): d (ppm) 16.8, 18.1, 26.1, 26.9, 40.0, 45.1, 51.3, 124.5, 126.6, 128.7, 128.8, 129.0, 129.2, 129.9, 132.0, 136.7, 139.5.

### Example 18

The following examples were synthesised according to Scheme 16:

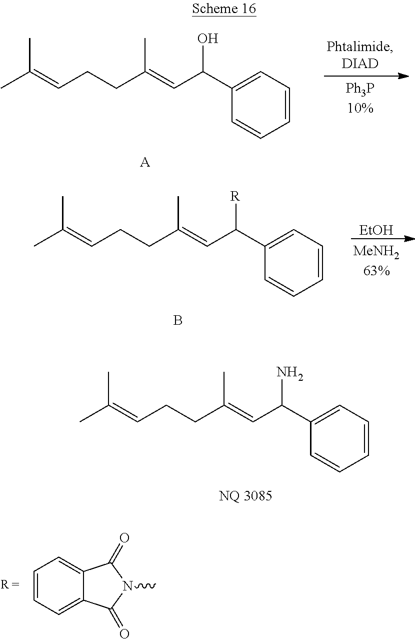

NQ 3085:

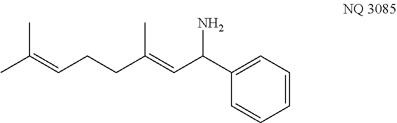 [SMILES: CC(C)=CCC/C(C)=C/C(N)c1ccccc1]

To a solution of 8.8 ml (15.7 mmol) 1.8 M phenyllithium in 10 ml anhydrous THF was added 2.0 g (13.1 mmol) geranyl aldehyde under argon atmosphere, cooling in ice water. The reaction was stirred for half an hour, recovering to room temperature. The reaction was quenched with water and 10 ml saturated ammonium chloride solution cooling in ice water. The mixture was extracted with ethyl acetate (2×20 ml) and dried over anhydrous sodium sulfate. (E)-3,7-dimethyl-1-phenylocta-2,6-dien-1-ol (A) (2.7 g, 90%) was obtained by flash column chromatography (20% ethyl acetate in hexanes).

To a solution of 1.1 g (4.3 mmol) triphenylphosphine and 0.6 g (4.3 mmol) phthalimide in 30 ml anhydrous THF add 1.0 g compound 6 and 0.7 ml (4.3 mmol) diisopropyl azodicarboxylate under argon atmosphere. The reaction was stirred overnight before removing all the solvents next day. The residue was extracted with ethyl ether/hexanes (1/1, 2×15 ml). (E)-2-(3,7-dimethyl-1-phenylocta-2,6-dienyl)isoindoline-1,3-dione B (0.15, 10%) obtained by flash column chromatography (10% ethyl acetate+5% ethyl ether in hexanes).

To a solution of 0.5 g (1.4 mmol) (E)-2-(3,7-dimethyl-1-phenylocta-2,6-dienyl)isoindoline-1,3-dione (B) in 20 ml anhydrous ethanol add 1.0 ml (8.4 mmol) 8 N methylamine. The reaction was refluxed for 4 hours before removing the solvents. The mixture was filtered after dissolving in dichloromethane/hexanes (1:1). (E)-3,7-dimethyl-1-phenylocta-2,6-dien-1-amine NQ 3085 (0.2 g, 63%) was obtained by flash column chromatography (10% methanol+1% ethyl ether+0.5% triethylamine in dichloromethane).

The spectral data are as follows: ¹H NMR (700 MHz, CDCl₃) δ (ppm) 1.60 (s, br, 5H), 1.68 (s, 3H), 1.75 (s, 3H), 2.03 (m, 2H), 2.11 (m, 2H), 4.78 (d, J=9.1 Hz, 1H), 5.09 (m, 1H), 5.36 (dd, J=9.1, 1.0 Hz, 1H), 7.24 (t, J=7.3 Hz, 1H), 7.34 (m, 2H), 7.38 (d, J=7.5 Hz, 2H). ¹³C NMR (176 MHz, CDCl₃): δ (ppm) 16.6, 17.7, 25.7, 26.4, 39.6, 53.2, 124.0, 126.3, 126.7, 128.5, 129.4, 131.6, 135.8, 145.9.

### Example 19

The following examples were synthesised according to Scheme 17:

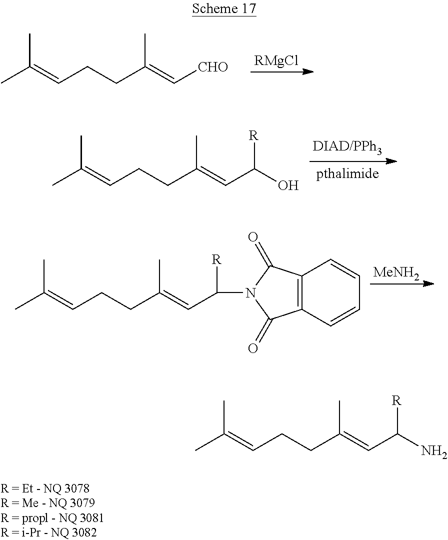

NQ 3078:

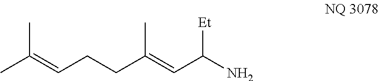

Grignard reagent ethylmagnesium chloride (6.5 mL, 13 mmol) was added to (E)-3,7-dimethylocta-2,6-dienal (1.52 g, 10 mmol) in dry THF (20 mL) at 0° C., and the mixture was stirred for 2 hours. The reaction was quenched with saturated NH₄Cl, and the mixture was extracted with EtOAc. The organic layer was dried with Na₂SO₄, and concentrated. The residue was purified with flash chromatography to afford (E)-5,9-dimethyldeca-4,8-dien-3-ol (1.69 g, 93%) as a colorless oil.

The spectral data are as follows: ¹H NMR (700 MHz, CDCl₃) δ (ppm) ¹H NMR (500 MHz, CDCl₃) δ (ppm): 0.93 (t, J=7.46, 3H), 1.45 (br, 1H), 1.47-1.53 (m, 1H), 1.63-1.71 (m, 4H), 1.73 (s, 6H), 2.06-2.10 (m, 2H), 2.13-2.17 (m, 2H), 4.31-4.36 (m. 1H), 5.13 (t, J=7.0, 1H), 5.20 (d-d, J=8.7, J=1.0, 1H; ¹³C NMR (125 MHz, CDCl₃): 9.78, 16.66, 17.74, 25.74, 26.40, 30.59, 39.62, 70.06, 123.95, 127.72, 131.72, 138.75.

Diisopropyl azodicarboxylate (DIAD, 1.28 mL, 6.5 mmol) was added to the solution of (E)-5,9-dimethyldeca-4,8-dien-3-ol (910 mg, 5 mmol) phthalimide (956 mg, 6.5 mmol) and PPh3 (1.73 g, 6.5 mmol) in dry THF (40 mL) at room temperature for 4 h. The reaction was quenched with brine, and the mixture was extracted with EtOAc. The organic layer was dried with Na₂SO₄, and concentrated. The residue was purified with flash chromatography to afford (E)-2-(5,9-dimethyldeca-4,8-dien-3-yl)isoindoline-1,3-dione (900 mg, 58%).

The spectral data are as follows: ¹H NMR (700 MHz, CDCl₃) δ (ppm): 0.89 (t, J=7.4, 3H), 1.57 (s, 3H), 1.64 (s, 3H), 1.71 (s, 3H), 1.91-1.94 (m, 1H), 1.98-2.04 (m, 3H), 2.07-2.09 (m, 2H), 4.89-4.92 (m, 1H), 5.06 (t, J=1.2, 1H), 5.09-5.11 (d-d, J=9.3, J=1.1, 1H), 7.70-7.71 (m, 2H), 7.82-7.83 (m, 2H); ¹³C NMR (175 MHz, CDCl₃): 11.00, 16.56, 17.69, 25.65, 26.25, 26.34, 39.43, 50.80, 122.79, 123.04, 123.89, 131.63, 132.08, 133.73, 139.60, 168.30.

(E)-2-(5,9-dimethyldeca-4,8-dien-3-yl)isoindoline-1,3-dione (540 mg, 1.74 mmol) and MeNH₂ (8 M in EtOH, 1.1 mL, 8.8 mmol) were stirred in ETOH (5 mL) at 70° C. for 3 h. The solution was concentrated, and 20 mL of hexanes was added to the mixture. The solid was filtered and washed with ether. The filtrate was concentrated and purified with flash chromatography to afford NQ 3078 (300 mg, 95%) as an oil.

The spectral data are as follows: ¹H NMR (700 MHz, CDCl₃) δ (ppm): 0.90 (t, J=7.5, 3H), 1.30-1.35 (m, 3H), 1.47-1.51 (m, 1H), 1.61 (s, 3H), 1.65 (d, J=1.3, 3H), 1.69 (d, J=0.6, 3H), 1.99-2.02 (m, 2H), 2.08-2.11 (m, 2H), 3.44-3.47 (m, 1H), 5.00 (d-d, J=8.9, J=1.0, 1H), 5.09-5.11 (m, 1H); ¹³C NMR (175 MHz, CDCl₃): 10.61, 16.47, 17.71, 25.71, 26.53, 31.37, 39.67, 50.76, 124.18, 130.10, 131.46, 135.68.

NQ 3079:

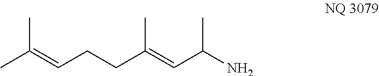

NQ 3079 was obtained in similar fashion by using methyl magnesium bromide.

The spectral data are as follows: ¹H NMR (700 MHz, CDCl₃) δ (ppm): 1.11 (d, J=6.4, 3H), 1.26 (br, 2H), 1.61 (s, 3H), 1.66 (s, 3H), 1.69 (s, 3H), 1.97-1.99 (m, 2H), 2.07-2.10 (m, 2H), 3.73-3.76 (m, 1H), 5.07-5.11 (m, 2H); ¹³C NMR (175 MHz, CDCl₃): 16.16, 17.62, 24.17, 25.59, 26.54, 39.48, 44.74, 124.15, 131.38, 131.63, 134.40.

NQ 3081:

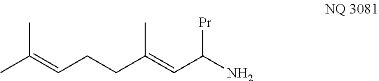

NQ 3081 was obtained in similar fashion by using propyl magnesium bromide.

The spectral data are as follows: ¹H NMR (700 MHz, CDCl₃) δ (ppm): 0.90 (t, J=7.2, 3H), 1.25-1.31 (m, 5H), 1.38-1.42 (m, 1H), 1.60 (s, 3H), 1.63 (d, J=1.3, 3H), 1.67 (s, 3H), 1.98-2.00 (m, 2H), 2.07-2.09 (m, 2H), 3.52-3.53 (m, 1H), 5.00 (d-d, J=9.0, J=1.0, 1H), 5.07-5.09 (m, 1H); ¹³C NMR (175 MHz, CDCl₃): 14.18, 16.38, 17.68, 19.42, 25.68, 26.47, 39.63, 40.79, 48.92, 124.15, 130.48, 131.43, 135.28.

NQ 3082:

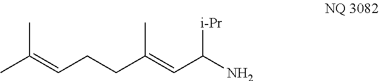

NQ 3082 was obtained in similar fashion by using isopropyl magnesium bromide.

The spectral data are as follows: ¹H NMR (700 MHz, CDCl₃) δ (ppm): 0.85 (d, J=6.8, 3H), 0.92 (d, J=6.8, 3H), 1.26 (br, 2H), 1.52-1.56 (m, 1H), 1.61 (s, 3H), 1.64 (s, 3H), 1.68 (s, 3H), 2.01-2.03 (m, 2H), 2.10-2.12 (m, 2H), 3.26-2.28 (m, 1H), 5.04 (d, J=9.2, 1H), 5.09 (t, J=6.8, 1H); ¹³C NMR (175 MHz, CDCl₃): 16.54, 17.70, 18.64, 19.02, 25.72, 26.49, 34.83, 39.81, 54.87, 124.24, 128.55, 131.42, 135.84.

### Example 20

The following examples were synthesised according to Scheme 18:

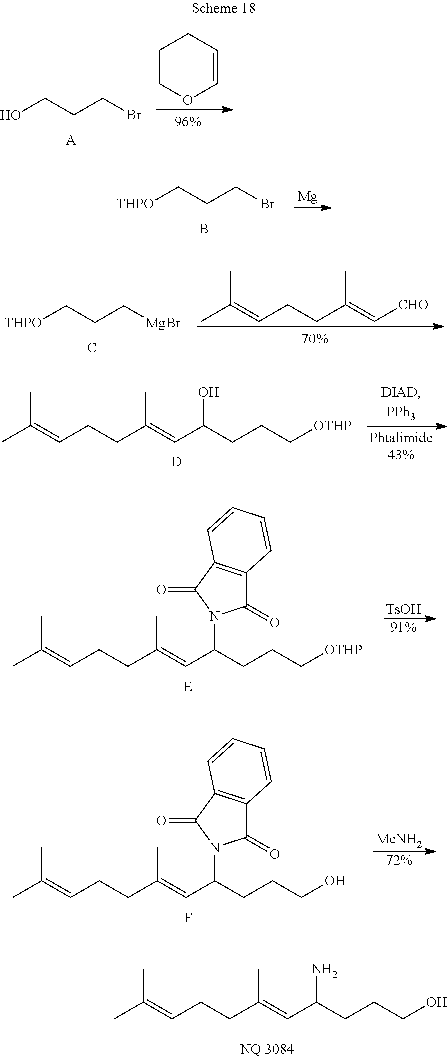

NQ 3084:

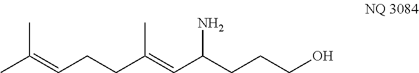

A solution of 3-bromo1-propanol (6.9 g, 50 mmol) and dihydropyran (5.04 g, 60 mmol) in methylene chloride (50 mL) containing TsOH (955 mg, 5 mmol) was stirred at room temperature for overnight h. The solution was diluted with hexane, washed with water, and dried over Na₂SO₄. Flash chromatography afforded the product B (10.7 g, 96%) as a clear oil.

The spectral data are as follows: ¹H NMR (700 MHz, CDCl₃) δ (ppm): 1.52-1.63 (m, 4H), 1.71-1.75 (m, 1H), 1.81-1.85 (m, 1H), 2.14-2.17 (m, 2H), 3.52-3.59 (m, 4H), 3.87-3.90 (m, 2H), 4.62 (s, 1H); ¹³C NMR (175 MHz, CDCl₃): 19.51, 25.43, 30.62, 30.75, 32.91, 62.29, 64.89, 98.92.

Magnesium powder (1.44 g, 60 mmol) in THF (20 mL) was activated by addition of 1,2-dibromoethane (0.2 mL) and stirring for 10 min. Then, bromide B (4.46 g, 20 mmol) in THF (30 mL) was added over 0.5 h at room temperature. The mixture was stirred for an additional 30 min. This Grignard reagent was cooled to 0° C. 3,7-dimethylocta-2,6-dienal was added and the mixture was allowed to gradually warm to room temperature for 3 h before a saturated ammonium chloride solution was added to quench the reaction. The mixture was extracted with hexane, washed with water, and dried over Na₂SO₄. The solvent was removed in a vacuum, and silica gel chromatography gave the product D (4.11 g, 70%) as a clear oil.

The spectral data are as follows: ¹H NMR (500 MHz, CDCl₃) δ (ppm): 1.53-1.61 (m, 8H), 1.63-1.74 (m, 10H), 1.82-1.87 (m, 1H), 2.02-2.05 (m, 2H), 2.10-2.16 (m, 3H), 3.43-3.47 (m, 1H), 3.53-3.58 (m, 1H), 3.78-3.83 (m, 1H), 3.87-3.92 (m, 1H), 4.39-4.44 (m, 1H), 4.62 (s, br, 1H), 5.11 (t, J=6.8, 1H), 5.21 (d, J=8.6, 1H); ¹³C NMR (125 MHz, CDCl₃): 17.72, 19.50, 19.51, 25.43, 25.72, 25.83, 25.94, 26.36, 30.62, 30.64, 34.80, 39.57, 62.20, 67.60, 67.64, 68.36, 68.42, 98.77, 123.97, 127.84, 131.66, 138.19, 138.21.

Diisopropyl azodicarboxylate (DIAD, 1.94 mL, 9.84 mmol) was added to the solution of D (2.24 g, 7.57 mmol) phthalimide (1.45 g, 9.84 mmol) and PPh₃ (2.58 g, 9.98 mmol) in dry THF (40 mL) at room temperature for 4 h. The reaction was quenched with brine, and the mixture was extracted with EtOAc. The organic layer was dried with Na₂SO₄, and concentrated. The residue was purified with flash chromatography to afford E (1.38 g, 43%).

A solution of THP ether E (500 mg, 1.18 mmol) and TsOH (23 mg, 0.12 mmol) in ethanol (10 mL) was stirred at 50° C. for 5 h. The reaction mixture was diluted with water, extracted with ethyl acetate, washed with water, and dried over Na₂SO₄, and the solvent was evaporated. This crude was used to the next step without any further purification.

F (280 mg, 0.82 mmol) and MeNH₂ (8 M in EtOH, 1.0 mL, 8 mmol) were stirred in EtOH (5 mL) at 70° C. for 3 h. The solution was concentrated, and 20 mL of hexanes was added to the mixture. The solid was filtered and washed with ether. The filtrate was concentrated and purified with flash chromatography to afford NQ 3084 (120 mg, 72%) as an oil.

The spectral data are as follows: ¹H NMR (500 MHz, CDCl₃) δ (ppm): 1.45-1.52 (m, 1H), 1.54-1.64 (m, 5H), 1.65 (d, J=1.4, 3H), 1.68-1.74 (m, 4H), 1.98-2.01 (m, 2H), 2.07-2.11 (m, 2H), 2.65 (s, br, 3H), 3.54-3.61 (m, 2H), 3.63-3.67 (m, 1H), 5.09-5.11 (m, 2H); ¹³C NMR (125 MHz, CDCl₃): 16.52, 17.88, 25.87, 26.63, 30.56, 36.39, 39.70, 49.37, 62.88, 124.15, 130.13, 131.77, 135.61

### Example 21

NQ 3083 was synthesised according to Scheme 19:

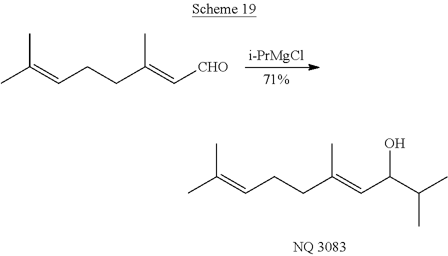

Grignard reagent iospropylmagnesium chloride (3.5 mL, 7 mmol) was added to (E)-3,7-dimethylocta-2,6-dienal (0.76 g, 5 mmol) in dry THF (20 mL) at 0° C., and the mixture was stirred for 2 hours. The reaction was quenched with saturated NH₄Cl, and the mixture was extracted with EtOAc. The organic layer was dried with Na₂SO₄, and concentrated. The residue was purified with flash chromatography to afford NQ 3083 (695 mg, 71%) as a colorless oil.

The spectral data are as follows: ¹H NMR (500 MHz, CDCl₃) δ (ppm): 0.88 (d, J=6.8, 3H), 0.97 (d, J=6.8, 3H), 1.45 (br, 1H), 1.63 (s, 3H), 1.67-1.73 (m, 7H), 2.05-2.08 (m, 2H), 2.11-2.14 (m, 2H), 4.09 (t, J=7.6, 1H), 5.12 (m, 1H), 5.21 (d-d, J=7.8, J=1.2, 1H); ¹³C NMR (125 MHz, CDCl₃): 16.84, 17.84, 18.15, 18.53, 25.85, 26.51, 34.64, 39.91, 73.76, 124.16, 126.47, 131.77, 139.07.

### Example 22

### Sodium (Na⁺) Channel Analysis in Rat DRG Neurons Using Whole Cell Patch-Clamp Techniques

Isolated DRG neurons were suspended in primary neuron basal media and placed on glass coverslips for incubation in humidified atmosphere of 5% CO₂ at 37° C. Coverslips carrying cells was transferred to the bath of an inverted microscope (Zeiss), continuously perfused with oxygenated artificial cerebro-spinal fluid (ACSF) containing (in mM) 124 NaCl, 2.5 KCl, 2 CaCl₂, 1 MgSO₄, 25 NaHCO₃, 1 NaH₂PO₄, and 10 glucose, at a rate of 2-3 ml/min. Recording of whole-cell membrane currents were made at room temperature. Recording pipette (4-6 MS2) was filled with internal solution containing (in mM) 145 K-gluconate, 5 NaCl, 1 MgCl₂, 0.2 EGTA, 10 HEPES, 2 Mg-ATP, 0.1 Na-GTP, and 10 phosphocreatine. To isolate Na⁺ currents, DRG neurons were superfused with ACSF containing tetraethylammonium chloride (TEA) 5 mM, cesium chloride (CsCl) 100 μM and cadmium chloride (CdCl) 1 mM, to block potassium and calcium currents. NQ compounds were freshly dissolved in ASCF containing TEA, CsCl and CdCl, prior application via the bath.

For recording Na⁺ currents, cells were held at −60 mV before applying a conditioning hyperpolarizing step (50 ms) to −90 mv to reactivate the voltage-gated Na^{+ channels. The conditioning pulse was followed by depolarizing (}150 ms) test pulses to 50 mV in 10 mV increments. Na⁺ currents were recorded in absence, after 3 min in presence of the drugs and after a recovery time of 3 min.

IC₅₀ values were measured and the observed ranges are shown in Table 2.

TABLE 2
IC₅₀ values for terpene analogues

| ID | | |
|---|---|---|
| Num- | | IC₅₀ |
| ber | Terpene analogue structure | range* |
| 2976 | 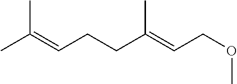 | C |
| 2977 | 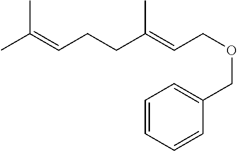 [SMILES: CC(C)=CCC/C(C)=C/COCc1ccccc1] | C |
| 2978 | 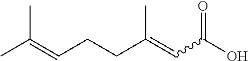 | E |
| 2980 | 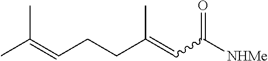 | B |
| 2981 | 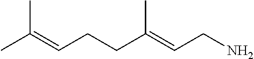 | A |
| 2982 | 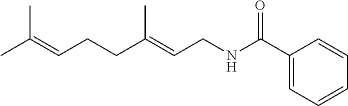 [SMILES: CC(C)=CCC/C(C)=C/CNC(=O)c1ccccc1] | C |
| 2983 | 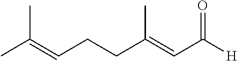 | C |
| 2984 | 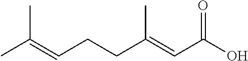 | C |
| 2985 | 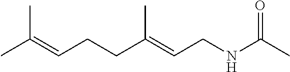 | C |
| 2986 | 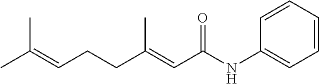 [SMILES: CC(C)=CCC/C(C)=C/C(=O)Nc1ccccc1] | D |
| 2987 | 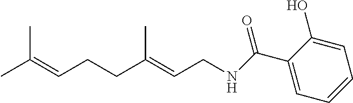 [SMILES: CC(C)=CCC/C(C)=C/CNC(=O)c1ccccc1O] | C |
| 2988 | 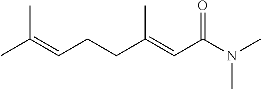 | C |
| 2990 | 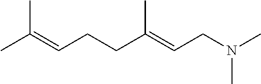 | C |
| 2991 | 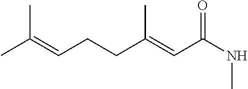 | C |
| 2992 | 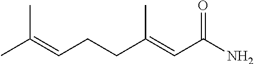 | B |
| 3000 | 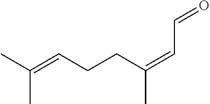 | D |
| 3001 | 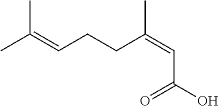 | E |
| 3007 | 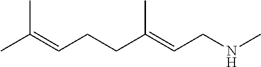 | C |
| 3045 | 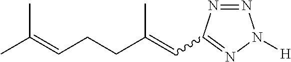 | C |
| 3047 | 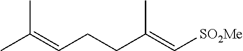 | C |
| 3050 | 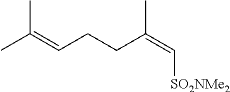 | B |
| 3051 | 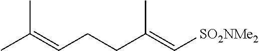 | B |
| 3052 | 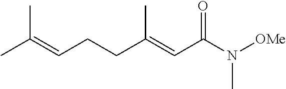 | B |
| 3053 | 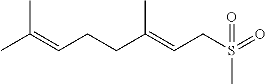 | C |
| 3054 | 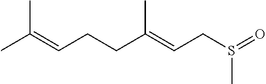 | C |
| 3055 | 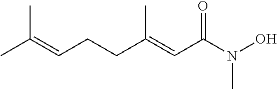 | B |
| 3057 | 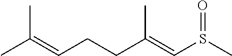 | C |
| 3060 | 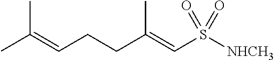 | C |
| 3061 | 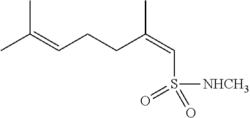 | B |
| 3062 | 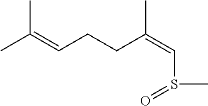 | C |
| 3063 | 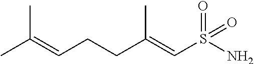 | C |
| 3064 | 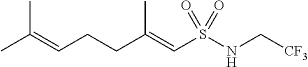 [SMILES: CC(C)=CCC/C(C)=C/S(=O)(=O)NCC(F)(F)F] | C |
| 3065 | 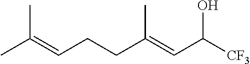 | B |
| 3066 | 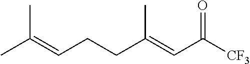 | C |
| 3067 | 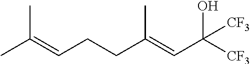 [SMILES: CC(C)=CCC/C(C)=C/C(O)(C(F)(F)F)C(F)(F)F] | B |
| 3069 | 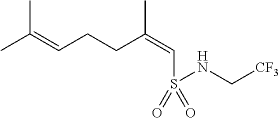 [SMILES: CC(C)=CCC/C(C)=C\S(=O)(=O)NCC(F)(F)F] | B |
| 3070 | 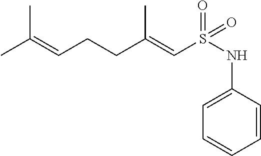 [SMILES: CC(C)=CCC/C(C)=C/S(=O)(=O)Nc1ccccc1] | B |
| 3071 | 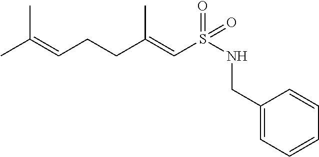 [SMILES: CC(C)=CCC/C(C)=C/S(=O)(=O)NCc1ccccc1] | B |
| 3078 | 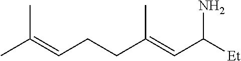 | A |
| 3079 | 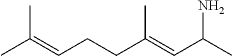 | B |
| 3081 | 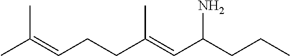 | A |
| 3082 | 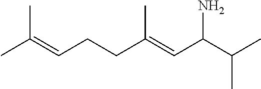 | A |
| 3083 | 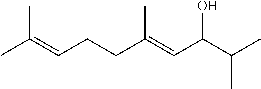 | C |
| 3084 | 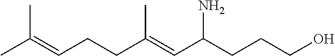 | B |
| 3085 | 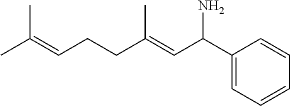 [SMILES: CC(C)=CCC/C(C)=C/C(N)c1ccccc1] | A |
| 3089 | 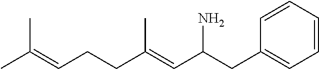 [SMILES: CC(C)=CCC/C(C)=C/C(N)Cc1ccccc1] | A |

*IC₅₀ ranges:
A = <0.1 mM
B = 0.1-1 mM
C = 1-5 mM
D = 5-10 mM
E = >10 mM

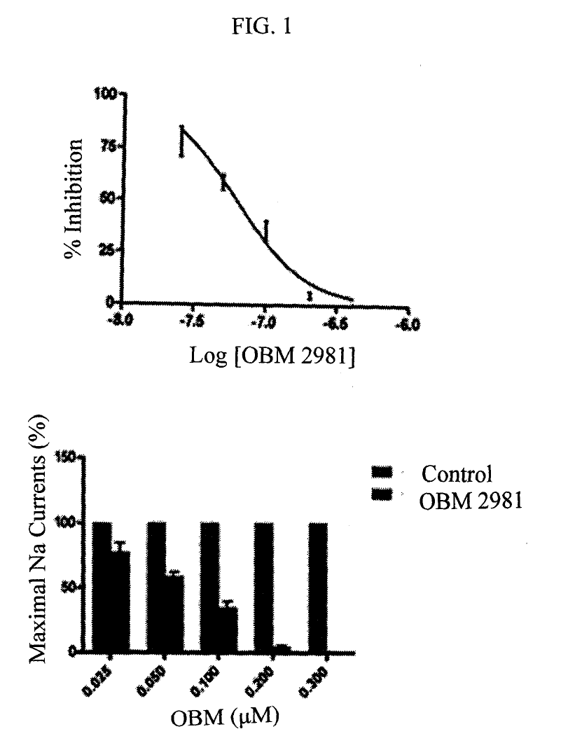

FIG. 1 shows a sodium channel patch clamp assay. The figure shows a representative inhibition curve for compound NQ 2981 and a plot of percentage sodium current versus concentration of NQ 2981 vs control. Calculated IC₅₀-62 nM. Note: “OBM 2981”=NQ 2981.

### Example 23

### Zebrafish Response Assay

Recent results indicate that certain zebrafish embryonic phenotypic readouts, reduced touch response and reduced spontaneous coiling, correlate with analgesic activity, providing an invaluable in vivo vertebrate preclinical bioassay for the identification and characterization of the activity of compounds capable of regulating neuropathic pain (data not shown).

Briefly, the ZEA assay involves applying essential oils, fractions or individual compounds to developmentally staged zebrafish embryos followed by monitoring of embryonic touch response/swim behaviour and evaluation of the dose response relationship for each substance. Using a four point scale to describe the embryonic behaviours (Table 4), initial analysis focused on monitoring and recording these changes and evaluating the level of bioactivity. The effective concentration to generate complete anaesthesia in 50% of the embryos (EC₅₀), were evaluated as follows:

Compounds are tested on developmentally staged AB “wild type” zebrafish embryos (54 hpf+/−2 hpf) at concentrations ranging between 10 and 400 μM.

Each compound is diluted in a 95% ethanol or DMSO carrier to create a working stock solution from which appropriate dilutions are made in standard embryo E3 media.

1000 μl of each concentration or appropriate carrier control are added to 10 wild type AB embryos in a single well of a 24 well plate, in duplicate.

The embryos are incubated for 90 min at 28° C. (optimal temperature for embryonic growth) in the diluted compound.

A four point scale (Table 4) is used to evaluate the touch response and swim behaviour for each embryo in all wells.

The effectiveness of the compound will be based on its ability to generate complete anaesthesia (scale: 1) in 50% of the embryos at a given concentration (EC₅₀).

The EC₅₀ values are calculated using GraphPad Prism® software to analyze the log (dose) response curves. These are shown in Table 3.

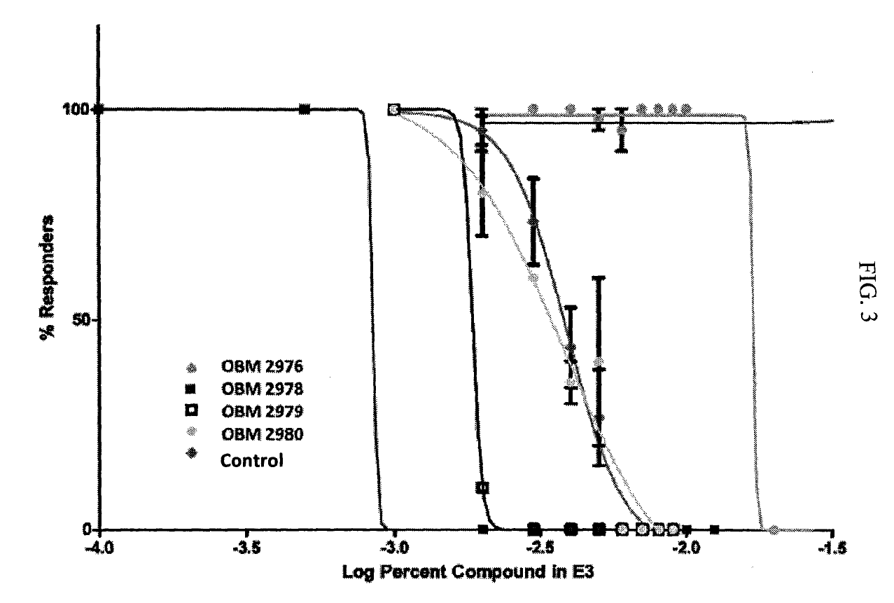

FIG. 3 shows a dose response curve of zebrafish embryo assay, percentage response versus percentage of compound present. Note: “OBM 2976”=NQ 2976; “OBM 2978”=NQ 2978; “OBM 2979”=NQ 2979; “OBM 2980”=NQ 2980.

TABLE 3
Measured EC₅₀ values.

| | | EC₅₀ |
|---|---|---|
| ID | Structure | (μM) |
| 2976 | 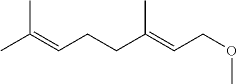 | 874 |
| 2977 | 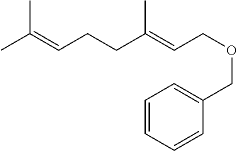 [SMILES: CC(C)=CCC/C(C)=C/COCc1ccccc1] | NA |
| 2978 | 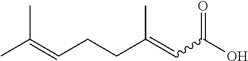 | 71.1 |
| 2980 | 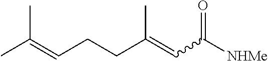 | 187 |
| 2981 | 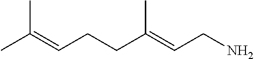 | 217.2 |
| 2982 | 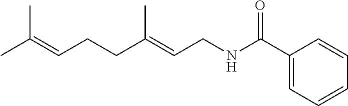 [SMILES: CC(C)=CCC/C(C)=C/CNC(=O)c1ccccc1] | 103.4 |
| 2983 | 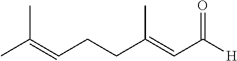 | 113.2 |
| 2984 | 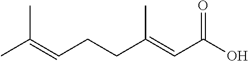 | >200 |
| 2985 | 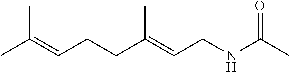 | 264 |
| 2986 | 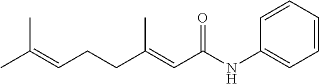 [SMILES: CC(C)=CCC/C(C)=C/C(=O)Nc1ccccc1] | 448.8 |
| 2987 | 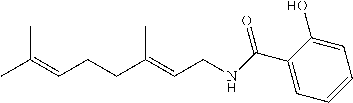 [SMILES: CC(C)=CCC/C(C)=C/CNC(=O)c1ccccc1O] | 134 |

TABLE 4
Four point scale representing 52-60hpf zebrafish embryonic behaviour.

| Scale | Behaviour |
|---|---|
| 4 | Normal embryonic swim behaviour and touch response |
| 3 | Burst touch response with no swimming |
| 2 | Twitch response to touch |
| 1 | No observable touch response or swim behaviour |

### Example 24

### TRPV1 Assay Protocol—Calcium Imaging

Briefly, cells are seeded into poly-L-lysine-coated, glass-bottom, 24-well plates (1×10⁵ cells/well) and incubated overnight under standard culture conditions to achieve the desired confluency. Culture media is removed and cells washed twice with HBS prior to incubation for 15 to 60 min at 37° C. with a labelling mixture comprised of Fura-2-AM and pluronic acid in HBS. Data collection occurs over an eight minute period and follows the same general sequence. Following loading, cells are stimulated by addition of 1 μM of capsaicin agonist for 2 min, after which a concentration series of the test sample (e.g., (0.5, 5, 10, 50 μg/ml) is added and imaging continued for an additional 5 min. Capsazepine (20 μM) serves as a known reference antagonist, while cells that are mock-treated or receive vehicle (e.g., DMSO) alone serve as negative controls. For imaging, plates are placed on the stage of an inverted epifluorescence microscope (e.g. Axiovert 200, Zeiss) equipped with a CCD digital camera (e.g., Axiocam MRm, Zeiss). For each well of the plate, a sequence of image pairs (excitation at 340 nm and 380 nm) are collected to capture intracellular calcium flux. Image sequences are analyzed in Image) (NIH) and average pixel intensities calculated for six representative cells in each test condition to achieve mean fluorescence. IC₅₀ are shown in Table 5.

TABLE 5
IC₅₀ values

| | | IC₅₀ |
|---|---|---|
| | | (μg/ |
| ID | Structure | mL) |
| 2976 | 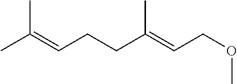 | — |
| 2977 | 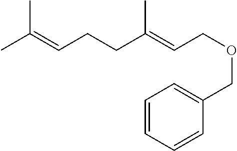 [SMILES: CC(C)=CCC/C(C)=C/COCc1ccccc1] | — |
| 2978 | 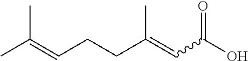 | — |
| 2980 | 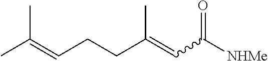 | >100 ug/ml |
| 2981 | 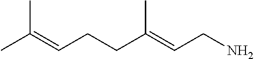 | — |
| 2982 | 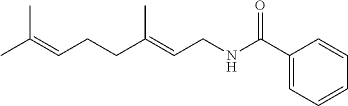 [SMILES: CC(C)=CCC/C(C)=C/CNC(=O)c1ccccc1] | — |
| 2983 | 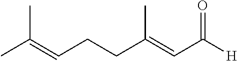 | 75-100 ug/Ml |
| 2984 | 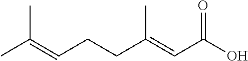 | — |
| 2985 | 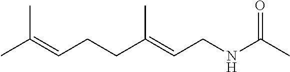 | — |
| 2986 | 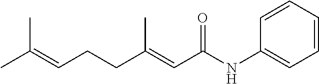 [SMILES: CC(C)=CCC/C(C)=C/C(=O)Nc1ccccc1] | — |
| 2987 | 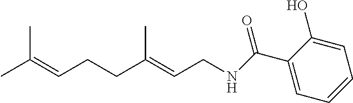 [SMILES: CC(C)=CCC/C(C)=C/CNC(=O)c1ccccc1O] | — |

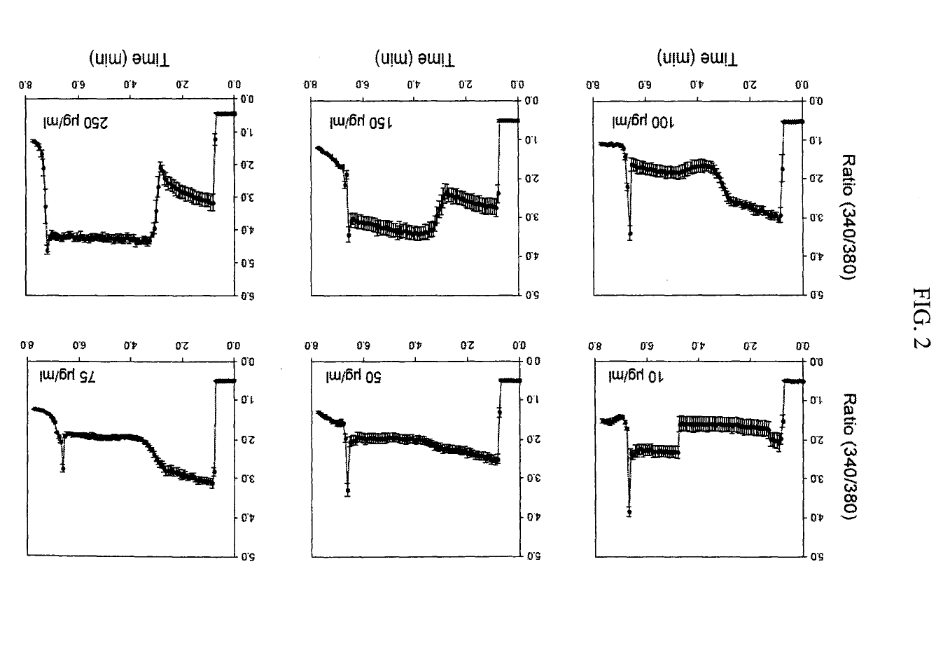

FIG. 2 shows Ca²⁺ imaging of NQ 2983 at various concentrations in the presence of HEK-TRPV cells. IC₅₀-493 μM.

To summarize, the results of the present studies demonstrate that terpenoid analogues of Formula 1 and 1a can be used in treatment of disorders of nerve transmission by restoring the balance between nerve excitation and inhibition. This can be achieved by affecting the activity of neuronal channels, such as sodium ion channels and TRP channels.

The compounds have been tested by bath application of known receptor antagonists and agonists to examine for changes in excitability and/or attenuation of ion channels, for the purpose of elucidating a mechanism of action. The compounds show significant ability to reduce membrane currents and early indication associated with the analgesic effects. In addition, patch clamp testing has shown that the compounds have a strong effect on sodium channel currents measured in dorsal root ganglion neurons. Voltage gated sodium channels are known to be relevant drug targets for neuropathic pain, as this family of ion channels governs the generation of action potential firing. (Josephine Lai, John C Hunter, Frank Porreca, The role of voltage-gated sodium channels in neuropathic pain Current Opinion in Neurobiology, Volume 13, Issue 3, June 2003, Pages 291-297).

Zebrafish embryos were tested, at various concentrations, to establish and identify conditions and phenotypic readouts (e.g., touch response, swim behavior) that could be used as an indicator of analgesic actively. Compounds in accordance with the presently disclosed and claimed inventive concept(s) were found to inhibit touch response in a dose dependent and reversible manner.

Further, compounds in accordance with the presently disclosed and claimed inventive concept(s) show various degrees of agonist and antagonist activity at the TRPV1 channel.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The presently disclosed and claimed inventive concept(s) being thus described, it will be obvious that the same may be varied in many ways. Such variations are not to be regarded as a departure from the spirit and scope of the presently disclosed and claimed inventive concept(s), and all such modifications as would be obvious to one skilled in the art are intended to be included within the scope of the following claims.

1. A method of treating a neurological condition comprising administering to a human or animal a therapeutically effective amount of a terpene analogue of Formula 1: 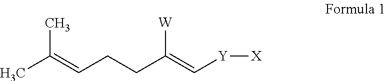
or a pharmaceutically acceptable isomer, salt, or ester thereof, wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocycle, wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl; and
W is H, a substituted or unsubstituted C₁ to C₂₀ alkyl, or a substituted or unsubstituted aryl.

2. The method of claim 1, wherein Y is CH₂, W is CH₃, and X is O—CH₃, O—CH₂-aryl, NH₂, N(H)—CH₃, N—(CH₃)₂, N(H)—C(═O)-aryl, N(H)—C(═O)—CH₃, N(H)—C(═O)-aryl(OH), SO₂Me, or SOMe.

3. The method of claim 1, wherein Y is C═O and X is H, OH, NH₂N(H)—CH₃, N—(CH₃)₂, N(H)-aryl, N(Me)OMe, N(Me)OH, or CF₃.

4. The method of claim 1, wherein the terpene analogue is a compound of Formula 1a: 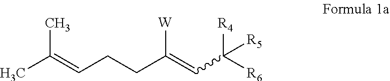
or a pharmaceutically acceptable isomer, salt, or ester thereof, wherein:
R⁴ is OH, alkoxyl, aryloxyl, —NH₂, —SO₂Aryl, SO₂alkyl, SOalkyl, —SO₂NHAryl, —NHSO₂Aryl, —NHalkyl, —N(alkyl)₂, —NHCO-Aryl; and
W, R⁵, and R⁶ are each independently H, a substituted or unsubstituted C₁ to C₂₀ alkyl, a substituted or unsubstituted aryl or a substituted or unsubstituted alkylaryl.

5. The method of claim 1, wherein:
Y is absent;
X is —C(═O)H, —C(═O)CF₃—COOH, —CH(OH)CF₃, —C(OH)(CF₃)₂, —C(═O)N(Me)OMe, C(═O)N(Me)OH, —CONHAryl, —CONH₂, —CONHAlkyl, —CON(Alkyl)₂—SO₂Aryl, —SO₂alkyl, SOalkyl, —SO₂NHAryl, —SO₂N(Aryl)₂, —SO₂N(Alkyl)₂, —SO₂NHalkyl, or SO₂NH₂; and
W is H, a substituted or unsubstituted C₁ to C₂₀ alkyl, a substituted or unsubstituted aryl or a substituted or unsubstituted alkylaryl.

6. The method of claim 1, wherein the terpene analogue is selected from the group consisting of:
(E)-1-methoxy-3,7-dimethylocta-2,6-diene,
(E)-((3,7-dimethylocta-2,6-dienyloxy)methyl)benzene,
3,7-dimethyloct-2,6-dienoic acid,
N,3,7-trimethylocta-2,6-dienamide,
(E)-3,7-dimethylocta-2,6-dien-1-amine,
(E)-N-(3,7-dimethylocta-2,6-dienyl)benzamide,
(E)-3,7-dimethylocta-2,6-dienal,
(E)-3,7-dimethylocta-2,6-dienoic acid,
(E)-N-(3,7-dimethylocta-2,6-dienyl)acetamide,
(E)-3,7-dimethyl-N-phenylocta-2,6-dienamide,
(E)-N-(3,7-dimethylocta-2,6-dienyl)-2-hydroxybenzamide,
(E)-N,N,3,7-tetramethylocta-2,6-dienamide,
(E)-N,N,3,7-tetramethylocta-2,6-dien-1-amine,
(E)-N,3,7-trimethylocta-2,6-dienamide,
(E)-3,7-dimethylocta-2,6-dienamide,
(Z)-3,7-dimethylocta-2,6-dienal,
(Z)-3,7-dimethylocta-2,6-dienoic acid,
(E)-N,3,7-trimethylocta-2,6-dien-1-amine,
5-(2,6-dimethylhepta-1,5-dien-1-yl)-2H-tetrazole,
(E)-2,6-dimethyl-1-(methylsulfonyl)hepta-1,5-diene,
(Z)—N,N,2,6-tetramethylhepta-1,5-diene-1-sulfonamide,
(E)-N,N,2,6-tetramethylhepta-1,5-diene-1-sulfonamide,
(E)-N-methoxy-N,3,7-trimethylocta-2,6-dienamide,
(E)-3,7-dimethyl-1-(methylsulfonyl)octa-2,6-diene,
(E)-3,7-dimethyl-1-(methylsulfinyl)octa-2,6-diene,
(E)-N-hydroxy-N,3,7-trimethylocta-2,6-dienamide,
(E)-2,6-dimethyl-1-(methylsulfinyl)hepta-1,5-diene,
(E)-N,2,6-trimethylhepta-1,5-diene-1-sulfonamide,
(Z)—N,2,6-trimethylhepta-1,5-diene-1-sulfonamide,
(Z)-2,6-dimethyl-1-(methylsulfinyl)hepta-1,5-diene,
(E)-2,6-dimethylhepta-1,5-diene-1-sulfonamide,
(E)-2,6-dimethyl-N-(2,2,2-trifluoroethyl)hepta-1,5-diene-1-sulfonamide,
(E)-1,1,1-trifluoro-4,8-dimethylnona-3,7-dien-2-ol,
(E)-1,1,1-trifluoro-4,8-dimethylnona-3,7-dien-2-one,
(E)-1,1,1-trifluoro-4,8-dimethyl-2-(trifluoromethyl)nona-3,7-dien-2-ol,
(Z)-2,6-dimethyl-N-(2,2,2-trifluoroethyl)hepta-1,5-diene-1-sulfonamide,
(E)-2,6-dimethyl-N-phenylhepta-1,5-diene-1-sulfonamide,
(E)-N-benzyl-2,6-dimethylhepta-1,5-diene-1-sulfonamide,
(E)-5,9-dimethyldeca-4,8-dien-3-amine,
(E)-4,8-dimethylnona-3,7-dien-2-amine,
(E)-6,10-dimethylundeca-5,9-dien-4-amine,
(E)-2,5,9-trimethyldeca-4,8-dien-3-amine,
(E)-2,5,9-trimethyldeca-4,8-dien-3-ol,
(E)-4-amino-6,10-dimethylundeca-5,9-dien-1-ol,
(E)-3,7-dimethyl-1-phenylocta-2,6-dien-1-amine,
(E)-4,8-dimethyl-1-phenylnona-3,7-dien-2-amine,
and combinations thereof.

7. The method of claim 1, wherein the terpene analogue is formulated for intravenous, topical, oral, intranasal, per rectal, intra muscular, intra dermal, intra vaginal, or subcutaneous administration.

8. The method of claim 1, wherein the neurological condition is pain.

9. The method of claim 8, wherein the pain is neuropathic pain.

10. A composition for treating a neurological condition, comprising a terpene analogue of Formula 1: 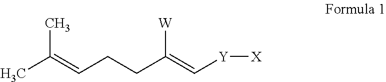
or a pharmaceutically acceptable isomer, salt, or ester thereof, wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocyclyl (for example, heteroaryl), wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl; and
W is H, a substituted or unsubstituted C₁ to C₂₀ alkyl, or a substituted or unsubstituted aryl.

11. The composition of claim 10, wherein Y is CH₂, W is CH₃, and X is O—CH₃, O—CH₂-aryl, NH₂, N(H)—CH₃, N—(CH₃)₂, N(H)—C(═O)-aryl, N(H)—C(═O)—CH₃, N(H)—C(═O)-aryl(OH), SO₂Me, or SOMe.

12. The composition of claim 10, wherein Y is C═O and X is H, OH, NH₂N(H)—CH₃, N—(CH₃)₂, or N(H)-aryl, N(Me)OMe, N(Me)OH, or CF₃.

13. The composition of claim 10, wherein the terpene analogue is selected from the group consisting of:
(E)-1-methoxy-3,7-dimethylocta-2,6-diene,
(E)-((3,7-dimethylocta-2,6-dienyloxy)methyl)benzene,
3,7-dimethyloct-2,6-dienoic acid,
N,3,7-trimethylocta-2,6-dienamide,
(E)-3,7-dimethylocta-2,6-dien-1-amine,
(E)-N-(3,7-dimethylocta-2,6-dienyl)benzamide,
(E)-3,7-dimethylocta-2,6-dienal,
(E)-3,7-dimethylocta-2,6-dienoic acid,
(E)-N-(3,7-dimethylocta-2,6-dienyl)acetamide,
(E)-3,7-dimethyl-N-phenylocta-2,6-dienamide,
(E)-N-(3,7-dimethylocta-2,6-dienyl)-2-hydroxybenzamide,
(E)-N,N,3,7-tetramethylocta-2,6-dienamide,
(E)-N,N,3,7-tetramethylocta-2,6-dien-1-amine,
(E)-N,3,7-trimethylocta-2,6-dienamide,
(E)-3,7-dimethylocta-2,6-dienamide,
(Z)-3,7-dimethylocta-2,6-dienal,
(Z)-3,7-dimethylocta-2,6-dienoic acid,
(E)-N,3,7-trimethylocta-2,6-dien-1-amine,
5-(2,6-dimethylhepta-1,5-dien-1-yl)-2H-tetrazole,
(E)-2,6-dimethyl-1-(methylsulfonyl)hepta-1,5-diene,
(Z)—N,N,2,6-tetramethylhepta-1,5-diene-1-sulfonamide,
(E)-N,N,2,6-tetramethylhepta-1,5-diene-1-sulfonamide,
(E)-N-methoxy-N,3,7-trimethylocta-2,6-dienamide,
(E)-3,7-dimethyl-1-(methylsulfonyl)octa-2,6-diene,
(E)-3,7-dimethyl-1-(methylsulfinyl)octa-2,6-diene,
(E)-N-hydroxy-N,3,7-trimethylocta-2,6-dienamide,
(E)-2,6-dimethyl-1-(methylsulfinyl)hepta-1,5-diene,
(E)-N,2,6-trimethylhepta-1,5-diene-1-sulfonamide,
(Z)—N,2,6-trimethylhepta-1,5-diene-1-sulfonamide,
(Z)-2,6-dimethyl-1-(methylsulfinyl)hepta-1,5-diene,
(E)-2,6-dimethylhepta-1,5-diene-1-sulfonamide,
(E)-2,6-dimethyl-N-(2,2,2-trifluoroethyl)hepta-1,5-diene-1-sulfonamide,
(E)-1,1,1-trifluoro-4,8-dimethylnona-3,7-dien-2-ol,
(E)-1,1,1-trifluoro-4,8-dimethylnona-3,7-dien-2-one,
(E)-1,1,1-trifluoro-4,8-dimethyl-2-(trifluoromethyl)nona-3,7-dien-2-ol,
(Z)-2,6-dimethyl-N-(2,2,2-trifluoroethyl)hepta-1,5-diene-1-sulfonamide,
(E)-2,6-dimethyl-N-phenylhepta-1,5-diene-1-sulfonamide,
(E)-N-benzyl-2,6-dimethylhepta-1,5-diene-1-sulfonamide,
(E)-5,9-dimethyldeca-4,8-dien-3-amine,
(E)-4,8-dimethylnona-3,7-dien-2-amine,
(E)-6,10-dimethylundeca-5,9-dien-4-amine,
(E)-2,5,9-trimethyldeca-4,8-dien-3-amine,
(E)-2,5,9-trimethyldeca-4,8-dien-3-ol,
(E)-4-amino-6,10-dimethylundeca-5,9-dien-1-ol,
(E)-3,7-dimethyl-1-phenylocta-2,6-dien-1-amine,
(E)-4,8-dimethyl-1-phenylnona-3,7-dien-2-amine,
and combinations thereof.

14. The composition of claim 10, wherein the terpene analogue is a compound of Formula 1a: 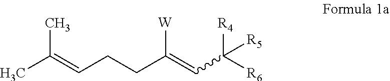
or a pharmaceutically acceptable isomer, salt, or ester thereof, wherein:
R⁴ is OH, alkoxyl, aryloxyl, —NH₂, —SO₂Aryl, SO₂alkyl, SOalkyl, —SO₂NHAryl, —NHSO₂Aryl, —NHalkyl, —N(alkyl)₂, or —NHCO-Aryl; and
W, R⁵, and R⁶ are each independently H, a substituted or unsubstituted C₁ to C₂₀ alkyl, a substituted or unsubstituted aryl or a substituted or unsubstituted alkylaryl.

15. The composition of claim 10, which is in a form for intravenous, topical, oral, intranasal, per rectal, intra muscular, intra dermal, intra vaginal, or subcutaneous administration.

16. The composition of claim 10, wherein the neurological condition is pain.

17. The composition of claim 16, wherein the pain is neuropathic pain.

18-25. (canceled)

26. A nerve transmission inhibitory composition, comprising a terpene analogue of Formula 1: 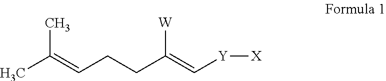
or a pharmaceutically acceptable isomer, salt, or ester thereof, wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocyclyl (for example, heteroaryl), wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl; and
W is H, a substituted or unsubstituted C₁ to C₂₀ alkyl, or a substituted or unsubstituted aryl.

27. The nerve transmission inhibitory composition of claim 26, wherein Y is CH₂, W is CH₃, and X is O—CH₃, O—CH₂-aryl, NH₂, N(H)—CH₃, N—(CH₃)₂, N(H)—C(═O)-aryl, N(H)—C(═O)—CH₃, N(H)—C(═O)-aryl(OH), SO₂Me, or SOMe.

28. The nerve transmission inhibitory composition of claim 26, wherein Y is C═O and X is H, OH, NH₂N(H)—CH₃, N—(CH₃)₂, or N(H)-aryl, N(Me)OMe, N(Me)OH, or CF₃.

29. The nerve transmission inhibitory composition of claim 26, wherein the terpene analogue is selected from the group consisting of:
(E)-1-methoxy-3,7-dimethylocta-2,6-diene,
(E)-((3,7-dimethylocta-2,6-dienyloxy)methyl)benzene,
3,7-dimethyloct-2,6-dienoic acid,
N,3,7-trimethylocta-2,6-dienamide,
(E)-3,7-dimethylocta-2,6-dien-1-amine,
(E)-N-(3,7-dimethylocta-2,6-dienyl)benzamide,
(E)-3,7-dimethylocta-2,6-dienal,
(E)-3,7-dimethylocta-2,6-dienoic acid,
(E)-N-(3,7-dimethylocta-2,6-dienyl)acetamide,
(E)-3,7-dimethyl-N-phenylocta-2,6-dienamide,
(E)-N-(3,7-dimethylocta-2,6-dienyl)-2-hydroxybenzamide,
(E)-N,N,3,7-tetramethylocta-2,6-dienamide,
(E)-N,N,3,7-tetramethylocta-2,6-dien-1-amine,
(E)-N,3,7-trimethylocta-2,6-dienamide,
(E)-3,7-dimethylocta-2,6-dienamide,
(Z)-3,7-dimethylocta-2,6-dienal,
(Z)-3,7-dimethylocta-2,6-dienoic acid,
(E)-N,3,7-trimethylocta-2,6-dien-1-amine,
5-(2,6-dimethylhepta-1,5-dien-1-yl)-2H-tetrazole,
(E)-2,6-dimethyl-1-(methylsulfonyl)hepta-1,5-diene,
(Z)—N,N,2,6-tetramethylhepta-1,5-diene-1-sulfonamide,
(E)-N,N,2,6-tetramethylhepta-1,5-diene-1-sulfonamide,
(E)-N-methoxy-N,3,7-trimethylocta-2,6-dienamide,
(E)-3,7-dimethyl-1-(methylsulfonyl)octa-2,6-diene,
(E)-3,7-dimethyl-1-(methylsulfinyl)octa-2,6-diene,
(E)-N-hydroxy-N,3,7-trimethylocta-2,6-dienamide,
(E)-2,6-dimethyl-1-(methylsulfinyl)hepta-1,5-diene,
(E)-N,2,6-trimethylhepta-1,5-diene-1-sulfonamide,
(Z)—N,2,6-trimethylhepta-1,5-diene-1-sulfonamide,
(Z)-2,6-dimethyl-1-(methylsulfinyl)hepta-1,5-diene,
(E)-2,6-dimethylhepta-1,5-diene-1-sulfonamide,
(E)-2,6-dimethyl-N-(2,2,2-trifluoroethyl)hepta-1,5-diene-1-sulfonamide,
(E)-1,1,1-trifluoro-4,8-dimethylnona-3,7-dien-2-ol,
(E)-1,1,1-trifluoro-4,8-dimethylnona-3,7-dien-2-one,
(E)-1,1,1-trifluoro-4,8-dimethyl-2-(trifluoromethyl)nona-3,7-dien-2-ol,
(Z)-2,6-dimethyl-N-(2,2,2-trifluoroethyl)hepta-1,5-diene-1-sulfonamide,
(E)-2,6-dimethyl-N-phenylhepta-1,5-diene-1-sulfonamide,
(E)-N-benzyl-2,6-dimethylhepta-1,5-diene-1-sulfonamide,
(E)-5,9-dimethyldeca-4,8-dien-3-amine,
(E)-4,8-dimethylnona-3,7-dien-2-amine,
(E)-6,10-dimethylundeca-5,9-dien-4-amine,
(E)-2,5,9-trimethyldeca-4,8-dien-3-amine,
(E)-2,5,9-trimethyldeca-4,8-dien-3-ol,
(E)-4-amino-6,10-dimethylundeca-5,9-dien-1-ol,
(E)-3,7-dimethyl-1-phenylocta-2,6-dien-1-amine,
(E)-4,8-dimethyl-1-phenylnona-3,7-dien-2-amine,
and combinations thereof.

30. The nerve transmission inhibitory composition of claim 26, wherein the terpene analogue is a compound of Formula 1a: 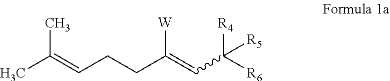
or a pharmaceutically acceptable isomer, salt, or ester thereof, wherein:
R⁴ is OH, alkoxyl, aryloxyl, —NH₂, —SO₂Aryl, SO₂alkyl, SOalkyl, —SO₂NHAryl, —NHSO₂Aryl, —NHalkyl, —N(alkyl)₂, or —NHCO-Aryl; and
W, R⁵, and R⁶ are each independently H, a substituted or unsubstituted C₁ to C₂₀ alkyl, a substituted or unsubstituted aryl or a substituted or unsubstituted alkylaryl.

31. The nerve transmission inhibitory composition of claim 26, which is in a form for intravenous, topical, oral, intranasal, per rectal, intra muscular, intra dermal, intra vaginal, or subcutaneous administration.
